SEQUENCE LISTING (14 sequences)

SEQ ID NO: 1 - DNA, 516 bp, Homo sapiens
gaagcggaggctgaattctgtgatctgcctcaaacccacagcctgggtagcaggaggaccttgatgctcctggcgcagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 2 - DNA, 498 bp, Homo sapiens
tgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 3 - DNA, 498 bp, Homo sapiens
tgtgatctgcctcaaacccacagcctgggtagcaggaggaccttgatgctcctggcgcagatgaggagaatctctcttttctcctgcttgaaggacagacatgactttggatttccccaggaggagtttggcaaccagttccaaaaggctgaaaccatccctgtcctccatgagatgatccagcagatcttcaacctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatga

SEQ ID NO: 4 - DNA, 23 bp, Homo sapiens
atggccttgacctttgctttact

SEQ ID NO: 5 - DNA, 29 bp, Homo sapiens
tcattccttacttcttaaactttcttgca

SEQ ID NO: 6 - DNA, 33 bp, Homo sapiens
gaagcggaggctgaattctgtgatctgcctcaa

SEQ ID NO: 7 - DNA, 31 bp, Homo sapiens
tcattccttacttcataaactttcttgcaag

SEQ ID NO: 8 - DNA, 44 bp, Homo sapiens
atctcgagaaaagagaagcggaggctgaattctgtgatctgcct

SEQ ID NO: 9 - DNA, 35 bp, Homo sapiens
aagcggccgctcattccttacttcttaaactttct

SEQ ID NO: 10 - DNA, 24 bp, Homo sapiens
gggaattctgtgatctgcctcaaa

SEQ ID NO: 11 - DNA, 23 bp, Homo sapiens
ttgcggccgctcattccttactt

SEQ ID NO: 12 - DNA, 29 bp, Homo sapiens
atctcgagaaaagatgtgatctgcctcaa

SEQ ID NO: 13 - DNA, 27 bp, Homo sapiens
tattctagatcattccttacttcttaa

SEQ ID NO: 14 - DNA, 28 bp, Homo sapiens
aagcggccgctcattccttacttcttaa

(19) United States
(12) Patent Application Publication
(10) Pub. No.: US 2006/0223145 A1
(43) Pub. Date: Oct. 5, 2006

(54) Method for producing recombinant human interferon alpha 2b polypeptide in pichia pastoris

(76) Inventors: Braj Bhushan Lohray, Ahmedabad (IN); Sarvagna Shah, Ahmedabad (IN); Hemal Pandit, Ahmedabad (IN); Megha Patel, Ahmedabad (IN)

Correspondence Address: NIXON & VANDERHYE, PC, 901 NORTH GLEBE ROAD, 11TH FLOOR, ARLINGTON, VA, 22203, US

(21) Appl. No.: 10/533,320

(22) PCT Filed: Nov. 1, 2002
(86) PCT No.: PCT/IN02/00216

§ 371 Date: Feb. 21, 2006

(51) Int. Cl. C12P 21/02; C07H 21/04; C12N 1/18; C07K 14/525; C12N 15/74

(52) U.S. Cl. 435/69.5; 435/254.23; 435/483; 530/351; 536/23.5

(57) ABSTRACT

This invention relates to a process for expression of recombinant Interferon-alfa2b polypeptide in yeast cells and its method of purification and formulation.

### FIELD OF THE INVENTION

The present invention relates to an immunomodulatory protein useful as antiviral and antitumor agent. Preferably, the present invention relates to a novel gene encoding human IFN alpha 2b protein The present invention also relates to novel polynucleotides used to isolate the novel gene; inserting the said gene in a suitable host; producing the culture of recombinant strain and stimulating expression of the heterologous polypeptide and its secretion. The invention also provides a method for high density fermentation process for production of interferon alfa 2b along with a suitable protein purification process for the same. Particularly, this invention relates to the preparation of human leukocyte IFN alpha 2b protein in high yields using a corresponding novel gene inserted in a recombinant Pichia pastoris strain.

### BACKGROUND OF THE INVENTION

Several human and animal interferons have been cloned, produced, purified and identified [Allen G., and Fantes F. H., Nature, 287, 408-411, (1980); Rubinstein M., et al, Arch. Biochem. Biophys, 210, 307-318, (1981); Cabrer B., Taira X, et al, J. Biol. Chem., 254, 3681-3684, (1979); Stewart W. E., II “The Interferon System,” Springer-Verlag, New York, 421, (1979); Kung H. F., et al, U.S. Pat. No. 4,672,108, (1987); Goeddel D. V., and Pestka S., U.S. Pat. No. 4,801,685, (Jan. 31, 1989); Goeddel D. V., and Pestka S., U.S. Pat. No. 4,810,645, (Mar. 7, 1989); Havell E. A., et al, Proc. Natl. Acad. Sci., USA, 72, 2185-2187, (1975); Pestka S., et al, Proc. Natl. Acad. Sci., USA, 72, 3898-3901, (1975); Pestka S., Arch Biochem Biophys., 221, 1-37 (1983); Bridgen P. J., et al, J. Biol. Chem., 252, 6585-6587, (1977); Rubinstein M., et al, Interferon: Properties and Clinical Uses, A. Khan, et al, Leland Fikes Foundation Press, Dallas, Tex., 45-55, (1980); Pestka S., Natural Products Isolation, G. H. Wagman and R. Cooper, eds., Elsevier, NY, 619 (1989); Knight E., Jr., Proc. Natl. Acad. Sci. U.S.A., 73, 520-523, (1976); Goeddel D. V., and Pestka S., European Patent Application 81105067.3 (1982); Pestka S., Methods in Enzymology, 119: 3-14, 14-23 (1986); Lawn R. M., et al, Proc. Natl. Acad. Sci. USA, 78, 5435-5439, (1981); Dworkin-Rastl E., et al, J. Interferon Research, 2, 575-585, (1982)].

During the last three decades several genes encoding IFN alpha subtypes have been identified [Goeddel D. V., et al, Nature 290:20-26 (1981); Valenzuela D., et al, Nature 313:698-700 (1985); Langer I. A., and Pestka S., J. Invest. Dermatol. 83, 128-136s, (1984)]. There are four main subtypes of interferon's, among which alpha or leukocyte interferon's are more common. These are also called as Type I interferon and are distinctly smaller in size, stable up to pH 2 and are glycoproteins. The major subtype gene of IFN alpha 2, is further subdivided into three classes which have been identified as (alpha 2a, alpha 2b, and alpha 2c). Generally in humans IFN alpha 2b is expressed more frequently. Currently the list of IFN subtypes includes nearly 20 genes, namely IFN-alpha 1a, -alpha 1b, -alpha 4a, -alpha 4b, -alpha 5, -alpha 6 etc [Streuli M., et al, Science, 209: 1343-7 (1980); Emanuel S. L., and Pestka, S., J. Biol. Chem., 268:12565-12569, (1993); Hosoi H., et al, International Society for Study of the Liver, Brighton, UK, 3-6th June, 113, Abstract, (1992); Desai M., et al, J. Interferon Res., 12: S138, (1992); Adolf G. R., et al, Biochem. J, 276:511-518, (1991)]. These subtypes are known to differ in their biological activities such as antiviral, anti-cell proliferation and NK-activation [Hu R., et al, J. Biol. Chem, 268: 12591-5, (1993)]. Relatively few protein-engineered variants of interferon have been reported [Sen G., et al, J. Virol., 50(2):445-450, (1984); Jones G., et al, Cancer, 57:1709-1715, (1986); Klingemann H. G., et at, Blood, 78(12):3306-3311, (1991)]. The amino acid sequence of human interferon alpha 2b has been reported [Lin L., et al, J. Gen. Virol., 39:125-130, (1978)] and the crystal structures of human Leukocyte Interferon subtypes have also been reported [Kung H. F., et al, U.S. Pat. No. 4,672,108, (1987)]. The biological actions reported for these proteins include antiviral, anti-proliferative and immunomodulatory properties [Baron S., et al., editors, The Interferon System: A Review to 1982—Part I and Part II, University of Texas Medical Branch, Galveston, 41 (1982); Baron S., et al, eds, Interferon: Principles and Medical Applications, The University of Texas Medical Branch at Galveston, Galvestan, 624 (1992); Pestka S., et al, Annu. Rev. Biochem. 56, 727-777, (1987)]. The example of various viral infections which may be treated using interferon include, but are not limited to: herpes simplex keratitis, acute hemorrhagic conjunctivitis, variacella zoster, cytomegalovirus infection, respiratory infections; including its uses in the treatment of genital warts [Bones R., Atkinson G., WO 98/23285, (1998).], hepatitis B [Gewert D., Salom C., et al, J Interferon Res., 13 (3), 227-231, (1993)] and psoriasis [Meritet J, et al, WO 01/42301, (2001)]. Other infections wherein treatment of interferon has been found to be useful include bacterial infections [ubin D., U.S. Pat. No. 4,762,705, (1988); Cummins J., et al, U.S. Pat. No. 5,830,456, (1988).

Type I interferon's are reported to be useful in treating cancers [Tanner D., et al, U.S. Pat. No. 5,028,422, (1991); Tanner D., et al, U.S. Pat. No. 5,256,410, (1993); Del B., U.S. Pat. No. 5,024,833, (1991); Wadler S., et al, U.S. Pat. No. 5,444,064, (1995); Wadler S., et al, U.S. Pat. No. 5,814,640, (1995); Peets E. A., et al, U.S. Pat. No. 5,002,764, (1991)], such as leukemias, basal carcinomas, squamous cell carcinomas, breast cancer, gastrointestinal malignancies, Kaposi's sarcoma, CML, B-cell and T-cell lymphomas, melanomas, renal cell carcinoma, ovarian, bronchogenic, bladder, and acute leukemias, malignant glioma and fibrosarcoma. Additionally, interferons are also used for actinic keratoses [Wong V. G., et al, U.S. Pat. No. 5,632,984 (1997)]. Carswell R. E., et al, EP 01/31789 (1985)], macular degeneration [Osther K. B., WO 98/06431 A2, (1998)], autoimmune disorders [Rusch L., WO 01/22970, (2001)] and diabetes [Bonnem E., U.S. Pat. No. 4,846,782 (1989)]. A method using IFN to selectively induce a programmed cell death (apoptosis) in cancerous cells is also provided [Carswell R. E., et al, EP 01/31789 (1985)]. Further, improved results have been shown when interferon therapy is given along with radiotherapy in treating cancer [Taylor et al, U.S. Pat. No. 5,831,062, (1998)]. The combination therapy of hTNF and human interferon has synergistic growth inhibitory or cytotoxic effect on tumors. Yet another application/use of the human interferon gene is to carry out gene therapy [Taylor et al U.S. Pat. No. 5,831,062, (1998)].

Their multifold actions on the immune system involve activation of macrophages, NK cells and intensifying the expression of various immunologically significant constituents of the cell membrane [McCabe M. M., WO 00/39280, (2000).].

As interferon have a species-specific activity, for its clinical use in humans; the protein should be obtained from genetic material directly related to the human interferon [Lin L., et al J. Gen. Virol., 39:125-130, (1978).]. Various protein formulations of IFN are in clinical use [Sen G., et al, J. Virol., 50(2):445-450, (1984); Jones G., et al Cancer, 57:1709-1715, (1986); Klingemann H. G., et. al., Blood, 78(12): 3306-3311, (1991); Physicians' Desk Reference, PDR, 47th Edition, 1993: pages 1078-1079; 1879-1881; 2006-2008; 2194-2201.]. Most of the approved interferons in clinical use are mixtures or individual species of human interferon alpha (Hu-IFN α).

In spite of such a wide applications, the clinical use of IFN has been limited due to limited availability of the protein. The process of interferon isolation and purification from the whole blood after appropriate stimulus remains unsatisfactory [Horowitz B., Methods in Enzymol., Academic Press, N.Y., 119:39-47 (1986).]. Suitable cell lines that can be used for the production of large amount of IFN alpha subtypes include leukocytes [Cantell K, et al, Methods in Enzymol., Academic Press, N.Y., 78:29-38, (1981); Khavlin T., et al, J. Leukocyte Biology, Annual Meeting Abstracts, Suppl. 3, Abstr. 137:36 (1992); Wheelock E. F., J. Bact. 92, 1415-1421, (1966); Ellis S. B., et. al., Mol. Cell. Biol., 9, 1316-1323, (1985); Morgensen K. E., et al., Pharmacol. Ther., 1, 369-381, (1977); Chirgwin J. M., et al., Biochemistry, 18: 5294, (1979); Stewart W. E., The Interferon System, Springer, Berlin, (1979).], Namalva cell (ATCC No. CRL-1432 etc.) [Dworkin-Rastl E., et. al., J. Interferon Research, 2, 575-585, (1982).], KG-1 cell etc. However, it has been reported that the proportion of different IFN subtypes synthesized upon induction in the each cell varies [Hiscott I., et al, Nucl. Acids. Res., 12, 3727-3746 (1984).]. Besides there are differences in properties of the protein and its native counterpart, which may affect the clinical efficacy and also there may be increased chances of negative host reactions, including fever, nausea, tissue necrosis, and psychopharmacological effects in the individual interferon subtypes [Adams F., et al, JAMA, 252(7):938-941 (1984); Wills R., et al Clin Pharmacol. Ther., 35(5):722-727 (May, 1984); Scott G., et al, J. Interferon Res., 1(1):79-85 (1980); Barouki F., et al, J. Interferon Res., 7:29-39 (1987)].

The advent of rDNA technology has made it possible to obtain IFN with relative ease and safety. Appropriate cloning and expression vectors which have been used for r-IFN production include bacterial, fungal, yeast and mammalian cellular hosts [Pestka S., Human Cytokines, Blackwell Scientific Publications 1-16 (1992); Biotherapy 10:59-86 (1997); U.S. Pat Nos. 4,897,471, 5,541,293 and 5,661,009], the relevant contents of each of which is hereby incorporated by reference. In the prior art, bacterial strains for production of interferon have been reported [Hauptmann R., et al., U.S. Pat. No. 5,710,027 (1998).]. Eukaryotic proteins produced in E. coil are sometimes nonfunctional, since glycosylation or other post-translational modifications do not occur because of lack of certain intracellular organelles in E. coli. Although exceptions are found, few recombinant interferon alpha have been cloned and expressed in E. coli and found to be biologically active [Streuli M., et. al., Science, 209: 1343-7 (1980); Goeddel D. V., et al., Nature, 287, 411-416, (1981); Nagata S., et. al., Nature, 284:316-310, (1980).].

Yeast cell has features such as ease of genetic manipulation and rapid growth characteristics like prokaryotic organism and biological characteristics typical of eukaryotic cell. This includes the sub-cellular machinery to carry our post-translational modification, which is desired. Commonly used yeast's include Hansenula polymorpha, S. cerevisiae and Pichia pastoris etc. strains, which are easier to work with variety of foreign genes. To prepare recombinant proteins, methylotrophic yeasts are most attractive candidates as it has certain genes, which are highly regulated and expressed under induced or de-repressed conditions [Nagata S., et. al., Nature, 284:316-310, (1980)].

The desired gene can either be isolated from a cDNA library of human leukocyte or obtained from genomic libraries that are commercially available. Alternatively, mRNA isolated from human leukocytes can be utilized to obtain gene of interest by the known methods in prior art [Desai M., et. al., J. Interferon Res., 12: S138, (1992) Cantell K, et al, Methods in Enzymol., Academic Press, N.Y., 78:29-38, (1981); Khavkin T., et al, J. Leukocyte Biology, Annual Meeting Abstracts, Suppl. 3, Abstr. 137:36 (1992); Wheelock E. F., J. Bact. 92, 1415-1421, (1966); Ellis S. B., et. al., Mol. Cell. Biol., 9, 1316-1323, (1985);]. Such methods include use of inducers such as viruses, natural or synthetic double-stranded RNA, intracellular microbes, microbial products and various other chemical agents.

The methods are available to isolate mRNA having an abundance of messages coding for human IFN alpha [Chirgwin J. M., et al., Biochemistry, 18: 5294, (1979); Stewart W. E., The Interferon System, Springer, Berlin, (1979); Hiscott I., et al, Nucl. Acids. Res., 12, 3727-3746 (1984).]. The mRNA isolated can be used to prepare cDNA of the present invention according to the methods described in prior art [Rubinstein M., et al., Methods in Enzymology, Academic Press, N.Y., 78A, 69-75, (1981).]. The cDNA can be converted into dsDNA using gene specific primers.

These dsDNA molecules can be cloned in suitable vectors and transformed into appropriate host such as E. coli or yeast Such a construct may include, an expression cassette comprising of a transcription promoter (T7, AOX1, Ga13) a gene encoding the polypeptide or protein of interest (e.g., dsDNA), and a transcription terminator (e.g., an AUG1 terminator, an AOX1 terminator, etc.). These elements should be operably linked so as to provide for transcription of the gene of interest and expression of a functional protein.

### OBJECTS OF THE INVENTION

The present invention aims to provide a method of producing novel recombinant DNA which encodes a polypeptide displaying immunological and biological activities of mature human interferon alpha 2b. Another objective of the present invention is to produce interferon alfa 2b by high density fermentation. Yet another objective of the present invention is to obtain IFN alpha 2b protein in a pure form. A further object of the present invention is to prepare a pharmaceutical composition comprising of the said recombinant human IPN alpha 2b protein or its pharmaceutically acceptable salt together with a pharmaceutically acceptable carrier or excipients.

A still further objective of the present invention is to prepare a pharmaceutically acceptable formulation of recombinant human IFN alpha 2b protein or its pharmaceutically acceptable salt for their uses in various diseases as mentioned herein.

### SUMMARY OF THE INVENTION

The present invention describes a novel DNA encoding human IFN alpha 2b protein and its method of production and purification. The process also involves novel oligonucleotides used as primers while isolating the novel gene. The appropriate gene after isolation is inserted into plasmid, which is further propagated in bacteria and later in yeast to give transformants having gene encoding for recombinant human IFN alpha 2b protein. The preferred cells for production of proteins for commercial use are methylotropic yeast. Using a new fermentation process, high-density cell culture of recombinant yeast is prepared by maintaining appropriate fermentation parameters. Later recombinant yeast cells are induced to produce desired protein in high yields. The said protein is purified by a novel purification process. The said purified protein is found to have all physiological, immunological and biochemical characteristics similar to mature human interferon alpha 2b protein.

### DETAILED DESCRIPTION OF THE INVENTION

The present invention will now be described in greater detail with reference to the accompanying drawings wherein:

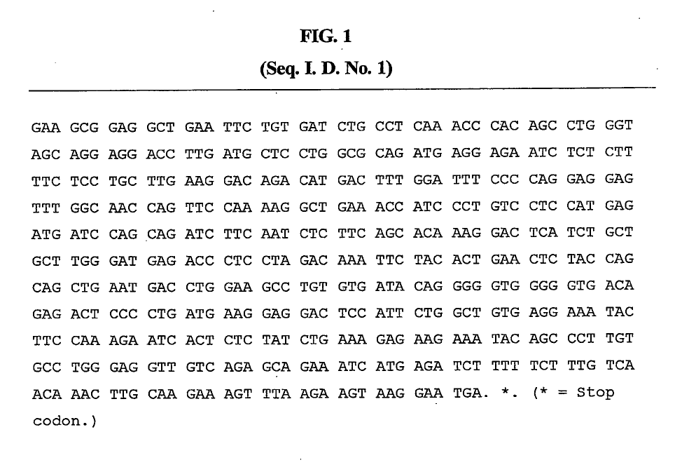

FIG. 1 shows the sequence (SEQ. ID. No. 1) of modified human interferon alpha 2b of the present invention.

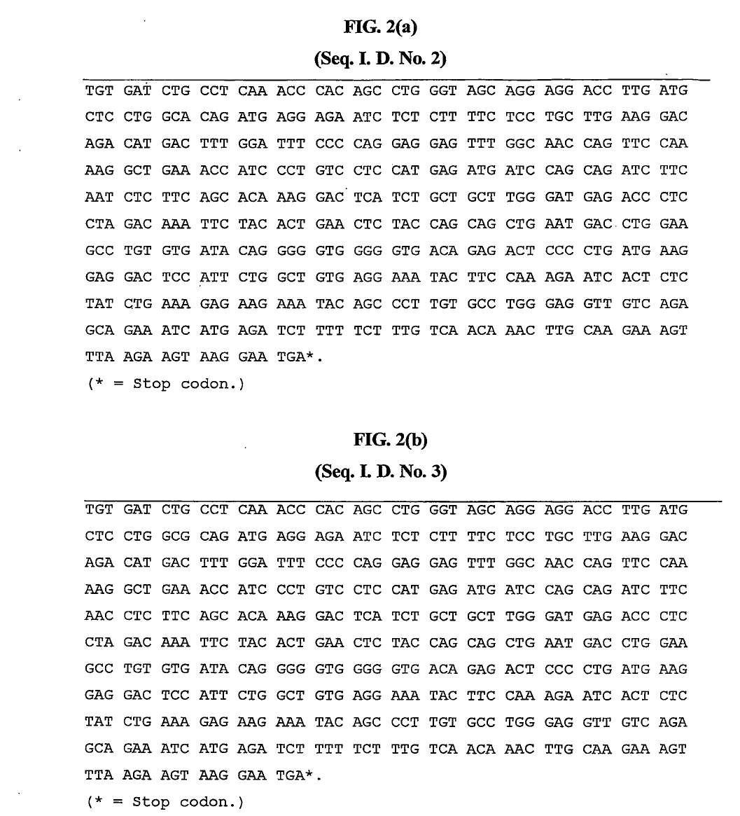

FIG. 2(a) Shows a sequence (SEQ. ID NO. 2) of IFN alpha 2b gene obtained from NCBI GenBank database wherein 57^{th} nucleotide is ‘A’ and 195^{th} nucleotide is ‘T’ in IFN alpha 2b gene.

FIG. 2(b) Shows the sequence (SEQ.ID.NO. 3) of human interferon alpha 2b of present invention which matches with the gene sequence deposited at the Gene Bank database (SEQ. ID. NO. 2) except at the 57^{th} position wherein instead of ‘A’, in the present invention there is ‘G’ and at the 19^{5th} position wherein instead of ‘T’, in the present invention there is ‘C’.

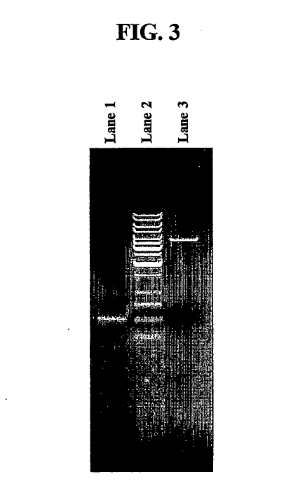

FIG. 3: Shows a restriction endonuclease digested and purified pPICZ alphaA DNA and IFN alpha 2b gene insert along with 1 kb ladder marker, on an agarose gel, stained by ethidium bromide (fluorescent dye). wherein, from left to right.

Lane 1: IFN alpha 2b insert (˜498 bp)

Lane 2: 1 kb DNA ladder

Lane 3: Purified ZBT alpha A DNA (3.6 Kbp)

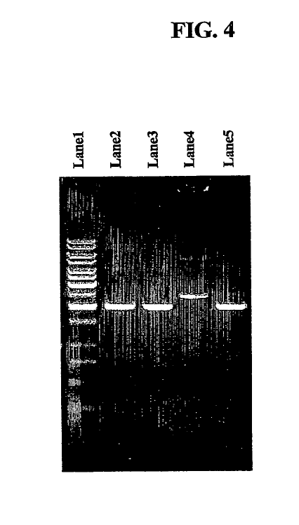

FIG. 4: Displays a recombinant pPICZ alphaA vector having IFN alpha 2b gene insert as shown by the mobility shift (Lane 4) on agarose gel wherein, from left to right.

Lane 1:1 kb DNA ladder.

Lane 2: ZBT alpha plasmid DNA

Lane 3: ZBT-IF 2.1 DNA

Lane 4: ZBT-IF 2.2 DNA

Lane 5: ZBT-IF2.3 DNA

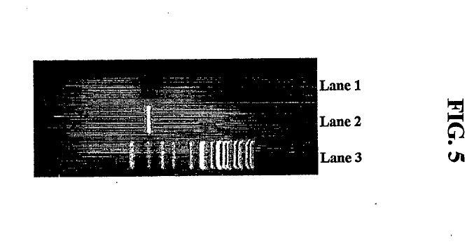

FIG. 5: Discloses the characterization of ZBT-IF 2.2 clones by PCR analysis prior to cloning into Pichia pastoris, wherein, from left to right.

Lane 1: Negative Control PCR product with gene specific primers where no amplification is observed

Lane 2: PCR product from ZBT-IF 2.2 plasmid DNA with gene specific primers (˜498 bp).

Lane 3: 1 kb DNA ladder

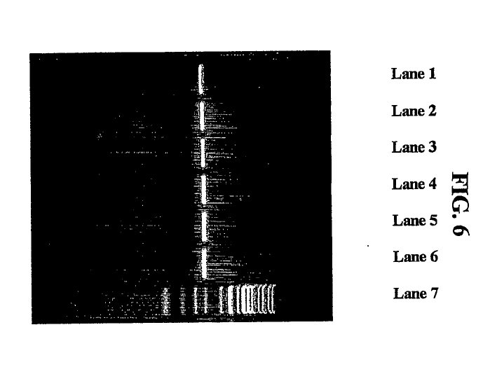

FIG. 6: PCR amplification of the IFN alpha 2b gene from the total genomic DNA isolated from Pichia pastoris clones (ZIF.2.2 series), wherein, from left to right:

Lane 1 to 6: PCR product from Pichia pastoris ZIF clone 2.2/1,2,3,4,5,6,7 clones genomic DNA with vector specific primers

Lane 7: 1 kb ladder marker.

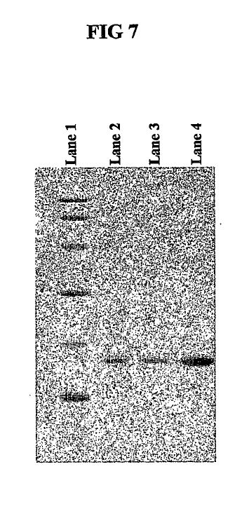

FIG. 7: shows SDS-PAGE of IFN alpha 2b polypeptide wherein, from left to right:

Lane 1: Low molecular weight marker.

Lane 2: European reference standard of IFN alpha 2b

Lane 3, 4: Show pure IFN alpha 2b protein of present invention.

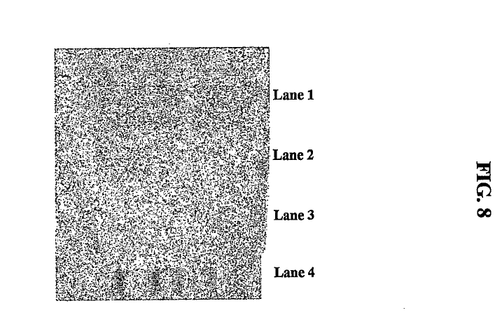

FIG. 8: shows Western blot of purified IFN alpha 2b protein produced by clone Pichia pastoris ZEF clone 2.2/14, wherein, from left to right:

Lane 1& 2: IFN alpha 2b protein of present invention

Lane 3: European Reference standard.

Lane 4: Prestained Low molecular weight Markers (BIO-RAD)

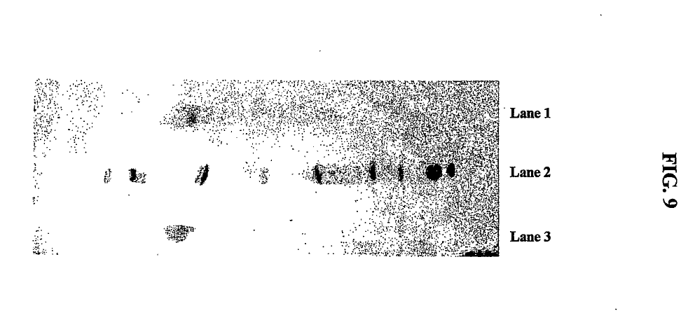

FIG. 9: shows Isoelectric focusing of purified IFN alpha 2b produced by clone Pichia pastoris ZIP clone 2.2/14 wherein, from left to right:

Lane 1: Pure IFN alpha 2b protein of present invention.

Lane 2: pI Markers (pI range 2.5 to 6.5)

Lane 3: European Reference standard

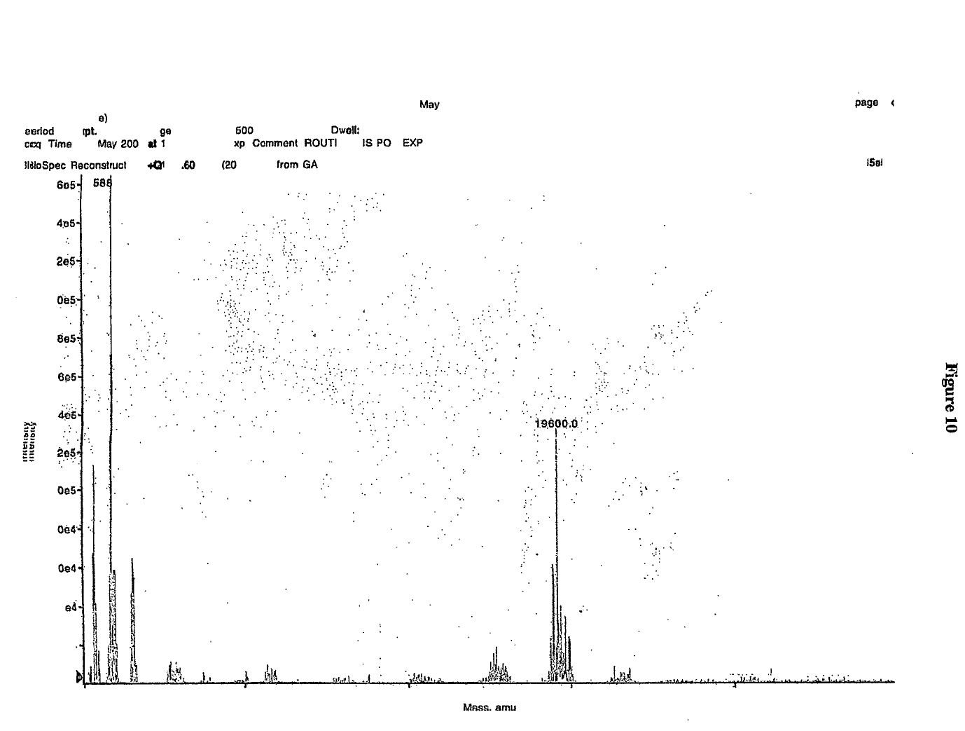

FIG. 10: shows LCMS of pure IFN alpha 2b produced by clone Pichia pastoris ZIF clone 2.2/14.

Novel DNA of the present invention encodes human interferon alpha 2b, specifically DNA comprising the base sequence as set forth in {FIG. 2b} (SEQ ID 3). The complete physical map of type-I interferon gene cluster and its location in chromosomes is known [Diaz M. O., et. al., J. Interferon Res., 11, S85, (1991); Owerback D., et. al., Proc. Natl. Acad. Sci. USA, 78, 3123-3127, (1981); Trent J. M., et. al., Proc. Natl. Acad. Sci. USA, 79, 7809-7813, (1982).].

In the gene sequence homology search, the novel DNA of the present invention was found to have >99% homology with corresponding part of some IFN subtypes deposited at NCBI GenBank database. The nearest matching known IFN alpha 2b sequence is described in FIG. 2a (SEQ ID 2). The novel DNA sequence of the present invention has a distinct nucleotide ‘Guanine’ at 57^{th} position, instead of ‘Adenine’ and ‘Cytosine’ at 195^{th} position instead of ‘Thymine’0 in comparison with the homologous sequences of human IFN alpha 2b gene. Further, another characteristic of this invention includes the protein encoded by said sequence has similarity to mature IFN alpha 2b protein in its primary structure. Derivatives of interferon, which are part of this invention and may be obtained by any one of the process disclosed herein, include various structural forms of the primary protein, which retain biological activity. Due to the presence of ionizable amino and carboxyl groups, for example, an interferon alpha 2b protein may be in the form of acidic or basic salts, or may be in a neutral form. Individual amino acids may be modified by oxidation or reduction.

The primary amino acid structure may be modified by forming covalent or aggregate conjugates with other chemical moieties, such as glycosyl groups, lipids, phosphate, acetyl, hydroxyacetyl, amido groups and the like, or by creating amino acid mutants.

Definitions:

In the description that follows, a number of terms are used extensively. The following definitions are provided to facilitate understanding of the invention.

As used herein, the terms “Interferon alpha 2b” or “IFN alpha 2b” refers to protein having amino acid sequences which are similar to the native mammalian interferon alpha 2b, which are capable of initiating response from Type I interferon receptor. The mature full-length human IFN-2b is usually a glycoprotein having a molecular weight of about 19.268 kilodaltons (kDa).

“Modified Interferon alpha 2b” refers to a nucleotide sequence having eighteen additional nucleotides at the 5′ end than the natural IFN alpha 2b.

The term “isolated” or “purified”, as used in the context of this specification to define the purity of interferon protein, means that the protein is substantially free of other proteins of natural or endogenous origin and contains less than about 1% by mass of protein contaminants residual of production processes.

“Recombinant” as used herein, means that a heterologous protein is derived from recombinant (e.g. microbial or mammalian) expression systems. “Microbial” refers to bacterial or fungal (e.g. yeast) expression systems. “Recombinant microbial product” defines a protein produced in a recombinant microbial expression system, which is essentially free of native endogenous substances.

Cloning Process:

Cloning of Interferon specific gene was initiated by stimulating specific RNA in human leucocytes based on the strategies outlined herein and by the procedures described in the literature (Cantell K, et al, Methods in Enzymol., Academic Press, N.Y., 78:29-38, (1981); Khavkin T., et al, J. Leukocyte Biology, Annual Meeting Abstracts, Suppl. 3, Abstr. 137:36 (1992); Wheelock E. F., J. Bact. 92, 1415-1421, (1966).). m-RNA was isolated and purified by using oligo dT columns according to known methods (Hiscott I., et al, Nucl. Acids. Res., 12, 3727-3746 (1984).). The purified m-RNA was used to prepare first strand of DNA by RT-PCR technique. The present process utilizes six sets of novel primers, which are specific for human IFN alpha. The nucleotide sequences of the first set of primers used to amplify the genes are shown in SEQ ID NOs.; 4 & 5; 6 & 7; of Table 2. The PCR products obtained with primers SEQ. ID NO.; 4 & 5 and 6 & 7 were used for cloning of modified human IFN alpha 2b gene in cloning vector. Another set of primers (SEQ ID NOs. 8 & 9/10 & 11), were used for subcloning of modified human IFN alpha 2b gene in suitable expression vector, which encodes for modified human IFN alpha 2b protein. The so obtained modified IFN alpha 2b clone was used further for cloning of mature human IFN alpha 2b using another set of primers (SEQ ID Nos. 12 & 13 or 12 & 14) to obtain a gene which encodes for human IFN alpha 2b protein. Suitable restriction endonuclease site such as EcoRI, XhoI site in the forward primer, while in reverse primer Xba I or Not I restriction sites may be incorporated in these oligonucleotides.

These oligonucleotide primers can be synthesized using the known techniques in the prior art [Broka C., et. al., Nucleic Acids Res., 8, 5461-5471 (1980); Beaucage, S. L., and Carothers, M. H., Tetrahedron Lett. 22, 1859-1862, (1981); Johnson B. A., et al., Biotechniques 8, 424-429, (1990)]. They can also be synthesized in automated oligonucleotide synthesizers for e.g., Oligo 1000 (Beckman, USA) using phosphoramidites and oligonucleotide synthesis kit. Alternatively, custom designed synthetic oligonucleotides may be purchased, for example, from Bangalore Genei (Bangalore, India).

The vector used to clone gene of interest may include plasmids derived from various sources such as E. coli, for e.g. pBR322, pBR325, pUC12, pUC13, M13mp18 etc.; Bacillus subtilis, for e.g. pUB110, pTP5, pC194, etc.; yeast for e.g. pSH19, pSH15, pPICZ alphaA, etc.; bacteriophages such as lambda phage: and other vectors such as pAT-11, pXT1, pRc/CMV, pRc/RSV, pcDNAI/Neo and the like [Maeda S., et al., Proc. Natl. Acad. Sci. USA 77, 7010-7013 (1980); 78, 4648 (1981); Raymond C. K, et al., BioTechniques 26(1):134-141 (1999)]. The preferred plasmid in the present invention is M13mp18 and pPICZ alphaA. The amplified PCR products were cloned in M13mp18 and propagated in E. coli JM109. A blue white screening was carried out to select the recombinant E. coli having the gene of present invention. The modified IFN alpha 2b was subcloned independently in pPICZ alphaA expression vector. The inserted DNA were amplified, isolated and sequenced. This modified clone was subsequently used for isolation and cloning of the mature IFN alpha 2b gene in a suitable expression vector. The so obtained clone of human IFN alpha 2b was isolated and sequenced. The nucleotide sequence was compared with the known gene sequences deposited at the NCBI GenBank database. This study lead to the conclusion that the unique gene encodes the human IFN alpha 2b subtype [Gewert D., Salom C., et. al., J Interferon Res., 13 (3), 227-231, (1993); Gabain A., et. al., Biotechnol. Appl. Biochem., 33(3):173, (2001).].

Besides plasmids, the expression vectors can either have genes acting as selective markers by imparting antibiotic resistance to the cells, such as G418 and other neomycin-type antibiotics (kanamycin resistance gene), or bleomycin/phleomycin-type antibiotics such as ZEOCIN (ble genes), as well as ampicilin resistance genes; or it may have gene for selective utilization of particular substrate for e.g. galactose, presence/absence of particular amino acid and the like. Preferably, selectable marker should be able to provide resistance in transformed yeast as well as bacteria. Other genes encoding dominant selectable markers are known to those of skill in the art [Romanos M., et al., DNA Cloning 2: Expression Systems, IRL Press, 2nd Ed., pages 123-167 (1995); Markie D., Methods Mol. Biol., 54:359 (1996); Pfeifer T. A., et al., Gene, 188:183, (1997); Tucker R. M., and Burke D. T., Gene, 199:25, (1997); Hashida O., et al., FEBS Letters, 425:117, (1998); Romanos, M., Curr. Opin. Biotechnol., 6:527-533 (1995); Laroche Y., et al., Biotechnology, 12:1119 (1994)]. One such auxotrophic marker in Pichia pastoris KM 71 is histidine gene. It is essential for the transformant or transfectant to have at least one copy of the DNA coding for the protein of interest.

The selection of promoter depends upon the host cell used for the transformation/recombinant gene expression. When the host cell is of an animal origin, suitable promoter can be used such as SV40 promoter, LTR promoter, CMV promoter, HSV-TK promoter and the like. When the host is E. coli, the suitable promoter can be trp promoter, lac promoter, lambda_{PL} promoter, lpp promoter, T7 promoter and the like. When the host is yeast, suitable promoters can be PHO5 promoter, PGK promoter, AOX1 and AOX2 promoter [Rodriguez L., et al., Yeast, 12:815 (1996); Saki Y., et al., U.S. Pat. No. 5,750,372.], GAP promoter [Waterham H. R., et al., Gene 186:37 (1997); Rosenberg S., et. al., U.S. Pat. No. 5,089,398.], ADH promoter and the like. Suitable promoters can be identified by its function according to known methods. The promoters of methylotrophic yeast, involved in methanol metabolism are particularly strong, and these are generally used to control the heterologous expression of proteins [Hollenberg C. P., et. al., EP 0173 378 (1991); Viader S. J., EP 0952158, (1999); Stroman D. W., et al., EP 0183 071 (1992);].

There are many possible host cells, which have or can express the gene of interest under the control of suitable promoters. These include different types of microorganism, mammalian cells and others exemplified by, Escherichia species, Bacillus species, yeast cells, animal cells, or higher eukaryotes and the like, capable of expressing an appropriate vector. Examples of Escherichia species include strains such as Escherichia coli DH1 [Low B., PNAS, 60: 160 (1968)], JM103 [Messing J., et al., Nucl. Acids Res., 9:309 (1981)], JA221 [Clarke L., et al., J. Mol. Biol., 120:517 (1978).], HB101 [Boyer H. W., J. Mol. Biol., 41: 459 (1969).], C600 [Genetics, 39:440 (1954)] and the like. Examples of Bacillus species are, for example, Bacillus subtilis MI114 [Yoshimura K., Gene, 24: 255 (1983)] and the like. Examples of suitable mammalian host cells includes, COS-7, monkey kidney cells [Gluzman Y., Cell 23:175, (1981)], L cells, C127, 3T3, Chinese hamster ovary (CHO), Hela and BHK cell lines and the like. Examples of yeast cells which can be used include, for example, Saccharomyces cerevisiae AH22, AH22R, NA87-11A, DKD-5D or 20B-12, Schizosaccharomyces pombe NCYC1913, Hansenula, Candida, or Pichia etc. It is preferable to use yeast cells to obtain the protein of this invention. It is more preferable to use methylotrophic yeast such as Pichia pastoris KM71 for the reason cited above.

It is advantageous to use an expression vector, which comprises of a secretory signal sequence to drive secretion of expressed heterologous proteins. A typical secretory peptide consists of about 20 amino acids and it has a hydrophobic core of 6 to 15 amino acids followed by hydrophilic amino acid residues. Suitable secretory signal sequences can be preferably derived from Saccharomyces cerevisiae or Pichia pastoris species and are exemplified by invertase gene (SUC2), acid phosphatase gene (PHO1 and PHO5), alkaline phosphatase gene, or alpha mating factor (MF.alpha.1), as well as a synthetic hybrid based on the PHOL sequence. The present invention has 85-89 amino acid long secretary signal, bearing kex2 and STE3 endopeptidase cleavage site as described by Brake et. al [Brake A. J., et al., PNAS, 81:4642-46 (1984).] and has yeast mating factor alpha as a preferred secretory signal.

The method for transformation depends upon the host cells selected in accordance to standard techniques. The prior art describes methods to carry transformation for Escherichia species [Cohen S. N., et al., PNAS, 69:2110 (1972); Reid J. D., et. al., Gene, 17: 107 (1982)], Bacillus species [Chang S., et al., Molecular & General Genetics, 168:111 (1979)], yeast cells [Becker D. M., et. al., Methods in Enzymology, 194: 182-187 (1991); Hinnen A., et. al., Proc. Natl. Acad. Sci. USA, 75:1929 (1978)] and animal cells [Chang S., et al., Molecular & General Genetics, 168:111 (1979); Hiroki Nakayama, et. al., Cell Engineering, 8:263-267 (1995) (Shujun Company); Graham F. L., Virology, Vol. 52, 456 (1973)] all of which are incorporated herein by reference. Methylotrophic yeast cells are preferred [Sudbery P., Curr. Opin. Biotech, 7:517 (1996); Gabain A., et. al., Biotechnol. Appl. Biochem., 33(3):173, (2001); Higgins D. R., and Cregg J. M., (eds.), Pichia Protocols, Methods in Molecular Biology, Humana Press, (Totowa, N.J.), 103: 249-261 (1998); Cregg J. M., et al., Mol. Cell. Biol., 5, 3376-3385, (1985); Cregg J. M., Gene Expression Systems: Using Nature for the Art of Expression, Academic Press, Inc., Fenandez and Hoeffler (Eds.), 157-191 (1999); Cregg J. M., et al., Mol. Cell. Biol. 9, 1316-1323, (1989).]. The most suitable method for transformation is LiCl method [Gietz R. D., et. al., Method in Molecular Biology, I. H. Evans eds., Humana Press (Totowa, N.J.)].

The transformants or transfectants wherein the expression vector carries at least one copy of functional DNA, can be isolated according to the aforementioned techniques. The culture of tranformants can be prepared as described below.

Escherichia or Bacillus species can be suitably cultured in a liquid culture medium, wherein the culture medium contains appropriate carbon, nitrogen, and mineral sources, necessary for the growth. The carbon sources may include glucose, dextrin, soluble starch, sucrose, etc. The nitrogen sources may include organic or inorganic substances such as ammonium salts, nitrates, corn steep liquor, peptone, casein, meat extracts, bean-cakes, potato extracts, etc. Examples of the minerals may include calcium chloride, sodium dihydrogen phosphate, magnesium chloride, etc. It is further allowable to add yeast's, vitamins, growth-promoting factors, etc. It is suitable that the pH of culture medium is at about 5 to 8.

In the case of yeast transformants, the culture medium used may include available commercial preparations and standard media found in literature. It is preferable to keep the pH of the culture medium in between 3 to 6, kept at about 25 to 35° C. for about 24 to 72 hrs along with aeration and stirring if required.

In the case of the transfectants of animal cells, the culture medium used may include MEM medium, DMEM medium, RPMI 1640 medium, which may contain 5 to 20% of fetal calf serum. The culture has pH of about 6 to 7 and is incubated at 30 to 40° C. for 15 to 60 hrs in 5-7% CO₂, environment. If required medium exchange, aeration and stirring may be applied.

In the present invention methylotrophic yeast, especially Pichia species is the preferred organism. The suitable transformants obtained described above were labeled as Pichia pastoris ZIF clone 2.2 series. The transformants obtained were verified for the presence of interferon alpha 2b gene. The transformants may have differences in expression levels of heterologous proteins, resulting due to factors such as the site of integration and copy number of the expression cassette and differences in promoter activity among individual isolates. Various screening methods to identify suitable transformants are available, which includes, for example, protein specific ELISA based assay or immunoblotting with IFN alpha 2b specific antibodies or specific HPLC assays. The recombinant clones of Pichia pastoris ZIF 2.2 series were screened for expression of the said proteins of this invention. From these recombinant clones, the clone Pichia pastoris ZIP 2.2/14 producing the human IFN alpha 2b protein was selected and the expressed protein was characterized further for its biological and immunological equivalence with the mature native human JFN alpha 2b.

The IFN alpha 2b protein expressed was confined on SDS-PAGE, (FIG. 7). Further the purified protein of this invention was compared with European Reference standard of IFN alpha 2b protein (CRS batch 2, catalogue no. I 0320301) by SDS-PAGE, IEF as a reference protein as described in FIG. 9.

Fermentation and Expression of Heterologous Protein:

A preferred expression host in this invention is methylotrophic yeast, examples of which includes suitable strains of Pichia methanolica, Hansenula polymorpha, Pichia pasioris and the like, more preferably Pichia pastoris strain as described earlier. The preferred Pichia pastoris transformants should carry at least one copy of an expression cassette comprising an alcohol-inducible promoter, secretory signal sequence, a novel DNA encoding for IFN alpha 2b protein, a transcription termination signal and a selection marker.

Broadly, the unique fermentation process of this invention comprises of producing a high-density cell-culture of novel Pichia pastoris ZIF 2.2/14 clone and expression df heterologous protein under suitable conditions. Various types of fermentation techniques such as batch, fed-batch, and continuous fermentation protocols are well known to those skilled in the art [Brock T. D., Biotechnology: A Textbook of Industrial Microbiology, Sinauer Associates, 2^{nd} Ed., (1989); Demain A. L. and Davies J. E., Manual of Industrial Microbiology and Biotechnology, 2^{nd} Ed., ASM Press, (1999); Hewitt C. J., et al., J. Biotechnol. 75:251 (1999)]. The necessary conditions, equipment's and materials required to carryout fermentation by any conventional fermentor are well known [Sherman F., Methods in Enzymology, Guthrie C. et al. (Eds.), Academic Press, N.Y., 194:14, (1991); Hollenberg C. P., and Gellissen G., Curr. Opin. Biotechnol., 8:554-560 (1997)]. Also, standard instrumentation is used to monitor various parameters such as temperature, pH, dissolved oxygen level, amount of nutrients such as carbon source/methanol and nitrogen. All equipment and additives are sterilized according to suitable methods known in prior art.

The typical fermentation protocol of the present invention provides conditions for high-density cell-mass build-up. The protocol has some characteristics of fed batch process of fermentation. The rate of addition of feed supply is related with the growth rate of cells, rate at which carbon and nitrogen are assimilated and also with CIH/N content of the cells.

In the preferred recombinant Pichia pastoris KM 71, the typical production process comprises of cells cultured in liquid medium at about 25° C. to 35° C., under aerated condition. Also it is known that in the case of Pichia pastoris the design of fermentor is an important factor during the process optimization [Ellis S. B., et. al., Mol. Cell. Biol., 9, 1316-1323, (1985); Villatte F., et. al., Appl. Microbiol. Biotechnol., 55(4):463-5, (2001); Morganti L., et al., Biotechnol. Appl. Biochem. 23:67 (1996); Stratton J., et al., Pichia Protocols, Higgins D. R., and Cregg J. M. (Eds.), Humana Press, Totowa,] as also described in the prior art [Brieley R. A., et al., International Publication No. WO 90/03431; Phillips A., et al, Methods in Enzymol., Academic Press, N.Y., 119:35-38 (1986).]. In the present invention, the high aeration requirement is provided by type, design & length of sparger, & by adjusting the agitation speed, air and oxygen supply based on dissolved oxygen concentration of fermentation broth and cell density. Exhaustion of glycerol leads to arrest of the logarithmic growth phase. At this point glycerol feed is initiated, and the feed rate is adjusted depending upon cell mass build up and utilization.

In the preferred fermentation process of the present invention, culture medium includes a suitable source for carbon such as glucose, glycerol sucrose etc., assimilable nitrogen such as nitrates, NH₄ as ammonical liquor, yeast nitrogen base etc., along with vitamins such as vitamin B₁₂, essential amino acids such as histidine, biotin, methionine etc., mineral supplements and trace metals such as manganese, mercury, iron and molybdenum salts, phosphates, sulfates etc. During fermentation, there can be single or multiple ingredients acting as a source for carbon to the growing cell culture. Suitable carbon sources include compounds, such as glycerol, glucose, fructose and the like, preferably glycerol. Alternatively, carbon source can include lower alcohols such as methanol, ethanol, propanol, isopropanol, butanol, isobutanol, and the like, preferably methanol. The examples of which include aqueous solution or syrups made using glucose or fructose, preferably aqueous solution. Glycerol may be used as the sole carbon source or 40% of glycerol can be mixed with aqueous solution containing other nutrients required by the yeast. Alcohol content of the media can range from about 0.1% to about 3%. For example, medium can contain alcohol about 0.5%, 1%, 2%, or 3%.

The fermentation is preferably conducted in a manner that the carbon source is a growth-limiting factor and thereby providing good conversion of the carbon source into higher cell mass buildup.

The assimilable nitrogen can be supplied using any nitrogen containing compounds capable of releasing nitrogen in a form that can be utilized by the yeast. The examples of nitrogen source includes organic or inorganic substances such as ammonium salts, nitrates, corn steep liquor, peptone, casein, meat extracts, bean-cakes, potato extracts, protein hydrolysates, yeast extract, urea, ammonium hydroxide and the like more preferably aqueous ammonia solution.

The media can also contain high level of inorganic salts, such as magnesium, maganese, copper, sodium, molybdenum, zinc, iron, potassium, calcium sulfate, phosphoric acid, orthophosphoric acid, sulfuric acid, boric acid and the like; vitamins such as biotin, thiamine and the like; protease inhibitors; amino acids such as histidine and the like; along with other trace nutrients and metals. Nevertheless, the medium can be supplemented with acid hydrolyzed casein (e.g., casamino acids or amicase) if desired to provide an enriched medium. In addition, media can also contain yeast's processing additives, growth-promoting factors, etc.

The pH range in the aqueous microbial ferment may be in the range of 4 to 7, preferably around 4.5 to about 6.5.

The preferred temperature during the fermentation is around 25° C.-30° C., preferably around 3° C.

The Pichia yeast requires aerobic conditions for growth, hence dissolved oxygen is required at all times during the fermentation. This may include supply of molecular oxygen in the form of air, oxygen enriched air or pure molecular oxygen itself so as to maintain the ferment with sufficient dissolved oxygen necessary to assist growth of cell. The overall aeration rates may vary from about 0.3 to 1.0 VVM (volume air per volume of ferment per minute). The level of dissolved oxygen in the culture medium may vary from a minimum of about 1% to about 100% saturation, more preferably about 30% to about 80% saturation, and most preferably about 20-60% saturation. During growth stage, the dissolved oxygen concentration may vary during the initial stages depending upon agitation (stirrer speed) in the fermentor.

To achieve high cell density in the fermentation, a fed batch fermentation protocol may be suitably modified. This may involve addition of suitable nutrients and carbon source. Alternatively, the batch can be modified by supplying booster feed of suitable nutrients from external source.

After the suitable growth phase, protein production may be induced using the suitable alcohol, selected from the group consisting of methanol, ethanol, propanol, isopropanol, butanol, and isobutanol, preferably methanol. Suitable time for starting the production phase can be between 48 to 110 hours of cell growth and the biomass achieved is approximately 100 to 200 g/L of wet weight. Induction of protein production is started by addition of methanol. Methanol is added in the concentration range of about 0.6 to 3.0% v/v preferably 0.8 to 2.8% v/v, which is monitored by gas chromatography. The production medium feed may deliver methanol in neat form or the alcohol may be diluted initially with suitable amounts of water or trace metal solution. Optionally along with a slow feed of methanol, glycerol may be provided for a short time to retain metabolic activity of the cells.

It is not necessary to continuously add methanol for the entire production phase of fermentation process in the present invention. According to a preferable embodiment of the present invention, a medium may contain 1 to 2% v/v methanol at the start of production phase of fermentation process. Production phase is monitored every 4-6 hours by sampling and examining various parameters which includes pH, OD, methanol concentration and increase in the concentration of the desired protein. Addition of methanol is controlled accordingly. Continuous or periodic addition of methanol is then started when the methanol concentration decreases to about 0.5% v/v or less, and for example, 0.2 to 0.5% v/v. In case the methanol concentration in the medium continues to decrease, eventually falling to 0% to 0.1% v/v the continuous addition may be started, and continued till methanol concentration of 2.0 to 3.0% v/v is attained.

During the time of methanol addition in this manner, the promoter is induced by methanol causing expression of the target gene, which encodes for IFN alpha 2b protein in the present invention. In addition to this, the added methanol may be partly used for the growth of the microorganisms. The preferred embodiment of this invention involves addition of medium or extra nitrogen source prior to induction, which may lead to growth of the microorganisms simultaneous to the protein production, over at least a certain period of time.

The present invention contemplates methods for producing a peptide or polypeptide in transformed Pichia, under the control of an alcohol-inducible promoter. Various fermentation protocols used to prepare high-density Pichia culture are outlined below and described in detail in the examples.

In one of the embodiments of the invention, fermentation was carried as fed batch as follows:

(a) Incubating the transformed Pichia cells in a complex medium, to produce a Pichia culture.

(b) Optionally, additional nitrogen source may be supplemented during growth stage.

(c) At the end of growth phase at about 65 to 78 hrs of elapsed fermentation time, the fermentation broth is centrifuged to separate cells from the spent medium. The cells are resuspended in the sterile production stage medium and transferred to fermenter aseptically.

(d) Alcohol feed is initiated, to induce the production of the recombinant protein of this invention.

Alternatively, in the present embodiment of the invention, culture media may be varied and may include defined salt media to grow the recombinant Pichia pastoris KM 71 ZIF 2.2/14, for expression of IFN alpha 2b as follows:

(a) Cultivation of the recombinant Pichia in a soluble defined salt medium by fed batch process, to produce a high-density cell biomass.

(b) Initiating an alcohol feed at about 90 to 110 hours of elapsed fermentation time, to induce the production of the recombinant protein of this invention.

In yet another embodiment of the present invention, the modification includes supplying additional carbon source to the Pichia culture, optionally other nutrients may also be supplied. Such a method for producing said protein comprises of the following steps:

(a) Incubating, the recombinant clone in a soluble defined salt medium.

(b) Initiating a glycerol feed at 60 to 80 hours elapsed fermentation time, wherein glycerol feed is sufficient to increase the biomass density, additionally sources of carbon, nitrogen and other nutrients may also be added.

(c) Stopping the glycerol feed at about 81 hrs to 95 hours-elapsed fermentation time.

(d) Initiating methanol feed at about 93 to 108 hours-elapsed fermentation time, wherein induction of heterologous protein synthesis by the recombinant Pichia pastoris is initiated.

In yet another variation of the above methods, the present invention provides methods for producing a recombinant IFN alpha 2b by recombinant Pichia pastoris KM 71 ZIF 2.2/14 comprising of the following steps:

(a) Initiating growth in soluble defined salt medium,

(b) Initiating a glycerol feed at 28 to 40 hours elapsed fermentation time, wherein sufficient glycerol feed along with suitable source of nitrogen and other nutrients are provided to increase the biomass density.

(c) Further growth of recombinant Pichia pastoris can be carried out in complex medium such as 1A, 1B, 1C, 1E (Table 1).

(d) Initiating an alcohol feed at about 93 hours to about 108 hours elapsed fermentation time, wherein the alcohol feed stimulates the production of the peptide or polypeptide by the recombinant Pichia pastoris.

Thus the fermentation medium (described in Table 1) is inoculated with a culture of recombinant Pichia pastoris containing Interferon alpha 2b gene in the presence of all required nutrients, oxygen, carbon and nitrogen source and all parameters of temperature, pH, dissolved oxygen are maintained as described in the embodiment and specified in the examples illustrated below to obtain high cell density and high yield of the desired protein in the fermentation broth.

Specific amounts and feeding rates are provided, however these specific amounts apply to the particular batch size and fermenter parameters exemplified. Those of skill in the art can vary these particular ingredients and amounts.

At the end of the production phase, the protein is isolated by conventional methods, either from the medium if the protein is secreted, or from the cells if it is not.

During the protein production phase, it is essential to minimize spurious proteolysis of recombinant peptides or polypeptides and various methods to inhibit in vivo proteolysis of the expressed heterologous proteins are known in the prior art [Zamost B., et. al., U.S. Pat. No. 6,258,559, (2001)]. In yeast, the major store of proteolytic activity is located within the lumen of the vacuolar compartment [Jones E. W., et. al., Methods in Enzymol., Academic Press, N.Y., 194:428 (1991).]. The common practices for minimizing the proteolytic degradation include saturating proteases by adding casamino acids or peptone to the culture medium and/or counteracting neutral proteases by reducing the pH level of the culture medium to about 3.0 [Gellissen G., et al., Gene Expression in Recombinant Microorganisms, Smith (ed.), Marcel Dekker Inc., 195-239 (1994); (U.S. Pat. No. 4,775,622,).].

TABLE 1
Different media used to culture recombinant
Pichia pastoris are as follows:

| No | Composition of the medium |
|---|---|
| 1A | BGY (complex medium) |
| | Peptone 20 g, Yeast Extract 10 g, Glycerol 10 ml, and phosphate |
| | buffer 0.5 M, pH 6.0 (100 ml) was added to each liter of medium |
| | prepared. After autoclaving 2 ml/L of biotin and histidine was |
| | added from stock solution. |
| 1B | BMY (complex medium) |
| | Peptone 20 g, Yeast Extract 10 g, Propylene Glycol 40 ml, and |
| | phosphate buffer 0.5 M, pH 6.0 (100 ml) was added to each liter |
| | of medium prepared. After autoclaving 2 ml/L of biotin and |
| | histidine was added from stock solution. |
| 1C | BMY (10×) (complex medium) |
| | Peptone 200 g, Yeast Extract 100 g, Propylene Glycol 400 ml, |
| | antifoam (10% dilution) 20 ml and phosphate buffer 1.0 M, pH = 6.0 |
| | (500 ml), was added to each liter of medium prepared. After |
| | autoclaving 2 ml/L of biotin and histidine was added from stock |
| | solution. |
| 1D | Defined salt Medium: |
| | To each liter of medium prepared, Glycerol 8.0 ml, phosphoric |
| | Acid 2.7 ml, Calcium sulfate 0.09 g, Potassium sulfate 1.8 g, |
| | potassium hydroxide 2.065 g, Magnesium sulfate. 7H₂O 1.5 g, trace |
| | metal solution A and B 4.4 ml each was added and pH was adjusted |
| | to 3.0 with aqueous ammonia solution. After autoclaving 2 ml/L of |
| | biotin and histidine was added from stock solution. |
| | Composition of trace metal solution A (PTM A) was Copper sulfate. |
| | 5H₂O 3.0 g, Maganese sulfate. 2H₂O 3.0 g, Sodium Molybdate 0.2 g, |
| | Boric acid 0.02 g, Zinc chloride 4.0 g and Sulfuric acid 5.0 ml. |
| | Composition of trace metal solution B (PTM B) was ferrous sulfate |
| | 5 g, and 2-3 drops of sulfuric acid. |
| 1E | BGYP medium |
| | Peptone 20 g, Yeast Extract 10 g, YNB without amino acid 6.7 g, |
| | Glycerol 20 ml and phosphate buffer 0.5 M, pH 6.0 (100 ml) was |
| | added to each liter of medium prepared. After autoclaving 2 ml/L |
| | of biotin and histidine was added from stock solution. |
| 1F | YPD agar |
| | Peptone 20 g, Yeast Extract 10 g, Dextrose 20 g, and agar agar 20 |
| | g (pH = 4.5) was added to each liter of medium prepared. |
| 1G | Luria Broth |
| | Casein Hydrolysate 10 g, Yeast extract 5 g, sodium chloride 5 g, |
| | and pH = 7.0 ± 0.2 was added to each liter of medium prepared. |
| 1H | BY (complex medium) |
| | Peptone 200 g, Yeast Extract 100 g, and phosphate buffer 0.5 M, |
| | pH = 6.0 to make up one liter of medium. After autoclaving 20 |
| | ml/L of biotin and histidine was added from stock solution. |

Stock solutions of certain compounds are prepared in appropriate
strengths as given below:
Biotin (2.0 g/L), Histidine (4.0 g/L), Ammonia (25% v/v 500 ml),
Antifoam (10% v/v 500 ml), Methanol (500 ml + 6.0 ml each of PTM
A & B), Glycerol feed (500 ml with 4.4 ml each of PTM A & B), Zeocin
Stock solution (100 mg/ml) and Tetracycline stock solution (25 mg/ml).
Each medium is sterilized by autoclaving, while the biotin,
histidine, tetracycline and zeocin stock solutions are sterilized by
filtration. Biotin and histidine stock solution is added at 2 ml/L
concentration after sterilization to each of above medium 1A, 1B,
1C, 1D and 1E.

Purification of Heterologous Protein Obtained from Transformed Piclia:

Prior art describes number of methods to isolate the recombinant proteins obtained from transformed yeast cells [Romanos M., et al., DNA Cloning 2: Expression Systems, IRL Press, 2nd Ed., pages 123-167 (1995); Trotta P. P., et al., Developments in Industrial Microbiology, Elsevier, Amsterdam, 53-64 (1987); Nagabhushan T. L. and Trotta P. P., Ullmann's Encyclopedia of Industrial Chemistry, A14, VCH, Weinheim, Federal Republic of Germany 372-374 (1989).]. Standard techniques, such as affinity chromatography, size exclusion chromatography, ion exchange chromatography, HPLC and the like can be used to purify the protein of interest.

Typically, secreted proteins can have purity anywhere in between 20-50%. The expressed polypeptide can be further purified to 90% purity; or even greater than 95% purity with respect to contaminating macromolecules, particularly other proteins, nucleic acids, and other infectious and pyrogenic agents. Polypeptides expressed by methylotrophic yeast may also be purified to a pharmaceutically pure state, which is greater than 99.0% pure.

In cases where proteins are secreted into culture media, advantage is of relatively lower contaminating substances, and the supernatant can be collected by known methods to isolate proteins [Berg K., Acta Path. Microbiol. Immunol. Scand., Section C, Suppl. 279, 1-136 (1982); Berg K, and Heron I., Methods in Enzymology, Academic Press, N.Y., 78, 487-499 (1981); Pestka S., and Rubinstein M., U.S. Pat. No. 4,289,690, (1981).]. This culture supernatant containing expressed protein can be purified by any one or more than one method in combination. The standard methods of protein purification are based on differences in the physicochemical characteristics of the proteins. The various methods are enlisted below:

i) solubility difference, examples of which include methods such as salting out, precipitation with solvents,

ii) differences in molecular size or weight, examples include dialysis, ultrafiltration, gel filtration and SDS-polyacrylamide gel electrophoresis,

iii) difference in the electric charge, examples include ion-exchange chromatography,

iv) methods utilizing specific affinity, examples include affinity chromatography,

v) methods utilizing a difference in the hydrophobic property, examples include reversed-phase high-performance liquid chromatography or Hydrophobic Interaction Chromatography and

vi) methods utilizing a difference in the isoelectric point examples include isoelectric electrophoresis, etc.

In order to obtain the native protein in its correctly folded state, it is preferable to use processes which avoids denaturation and precipitation steps.

Accordingly, the present invention is concerned with production and purification of homogeneous IFN alpha 2b using chromatographic techniques particularly ion exchange and concentrating the purified protein by ultrafiltration. The present invention aims to provide a large-scale protein purification process to achieve high degree of purity in an economical way.

Suitable chromatographic media include derivatized dextrans, agarose, cellulose, polyacrylamide, specialty silicas and the like, including PEI, DEAE, QAE, and Q derivatives. Examples of chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like, or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. Optionally, one can modify these supports with reactive groups such that proteins can link with amino, carboxyl, sulfhydryl, hydroxyl and/or carbohydrate moieties from the protein. Also, it is possible to engineer a tag onto the amino- or carboxyl-terminus of the recombinant protein to allow purification by affinity chromatography [125, 139-1401.

In cases where the protein thus obtained is in a free form, the free protein can be converted into a salt thereof by known methods or method analogous thereto. In case, where the protein thus obtained is in a salt form vice versa, the protein salt can be converted into a free form or into any other salt thereof by known methods or method analogous thereto. The suitable buffer solution may contain a protein-denaturing agent such as urea or guanidine hydrochloride or a surfactant such as Triton X-100 TM.

The process of this invention preferably elutes the interferon from ion exchange column by increasing the pH. Such pH increase can be obtained by applying a buffer solution to the column. Such process may involve applying a solution of the said crude interferon onto a column, such as eluting the adsorbed interferon from said column using a buffer solution, wherein suitable known techniques of the chromatography may be used, which may include for example salt gradient or pH; concentrating the eluate obtained from previous step in a suitable way; The purification process was carried out according to the following schemes:

Scheme 1:

a) reconditioning of the supernatant by dilution, pH adjustment, and filtration;
b) applying a solution of said crude interferon onto cation exchange column, preferably using a weak cation exchange such as carboxy methyl column (CM Sepharose FF, Amersham Biotech, Sweden.) and eluting the adsorbed interferon from said column using aqueous buffer solution;
c) applying the eluate resulting from above step (b) onto an anion exchange column, such as DEAE or Q Sepharose FF (Amersham Biotech, Sweden.) and eluting the adsorbed interferon from said column using a different aqueous buffer solution;
d) concentrating the eluate resulting from step c) by ultrafiltration provided with membrane of pore size 10,000 Dalton molecular cut off;
e) passing the concentrated protein resulting from step d) through gel filtration column, wherein the said column is equilibrated with buffer containing Tween-80 and EDTA to obtain homogeneous IFN alpha 2b.
   Scheme 2:
a) reconditioning of the supernatant by dilution, pH adjustment, and filtration;
b) applying a solution of said crude interferon onto cation exchange column, preferably using a weak cation exchange such as carboxy methyl column (CM Sepharose FF, Amersham Biotech, Sweden.) and eluting the adsorbed interferon from said column using aqueous buffer solution;
c) applying the eluate resulting from above step (b) onto an anion exchange column, such as DEAE or Q Sepharose FF (Amersham Biotech, Sweden.) and eluting the adsorbed interferon from said column using a different aqueous buffer solution;
d) Eluated aqueous solution from step c) containing IFN alfa 2b captured on to the Hydrophobic Interaction Chromatography column and eluted out using third aqueous buffer.
e) concentrating the eluate resulting from step d) by ultrafiltration provided with membrane of pore size 10,000 Dalton molecular cut off;
f) passing the concentrated protein resulting from step e) through gel filtration column, wherein the said column is equilibrated with buffer containing Tween-80 and EDTA to obtain homogeneous IFN alpha 2b.
   Scheme 3:
a) reconditioning of the supernatant by dilution, pH adjustment, and filtration;
b) applying a solution of said crude interferon onto cation exchange column, preferably using a weak cation exchange such as carboxy methyl column (CM Sepharose FF, Amersham Biotech, Sweden.) and eluting the adsorbed interferon from said column using aqueous buffer solution containing EDTA;
c) applying the eluate resulting from above step (b) onto an anion exchange column, such as DEAE or Q Sepharose FF (Amersham Biotech, Sweden.) and eluting the adsorbed interferon from said column using a different aqueous buffer solution containing EDTA;
d) concentrating the eluate resulting from step c) by ultrafiltration provided with membrane of pore size 10,000 Dalton molecular cut off;
e) passing the concentrated protein resulting from step d) through gel filtration column, wherein the said column is equilibrated with buffer containing Tween-80 and EDTA to obtain homogeneous IFN alpha 2b.
   Scheme 4:
a) reconditioning of the supernatant by dilution, pH adjustment, and filtration;
b) applying a solution of said crude interferon onto cation exchange column, preferably using a weak cation exchange such as carboxy methyl column (CM Sepharose FF, Amersham Biotech, Sweden.) and eluting the adsorbed interferon from said column using aqueous buffer solution containing EDTA;
c) applying the eluate resulting from above step (b) onto an anion exchange column, such as DEAE or Q Sepharose FF (Amersham Biotech, Sweden.) and eluting the adsorbed interferon from said column using a different aqueous buffer solution containing EDTA;
d) Eluated aqueous solution from step c) containing IFN alfa 2b captured on to the Hydrophobic Interaction Chromatography column and eluted out using third aqueous buffer.
e) concentrating the eluate resulting from step d) by ultrafiltration provided with membrane of pore size 10,000 Dalton molecular cut off;
f) passing the concentrated protein resulting from step e) through gel filtration column, wherein the said column is equilibrated with buffer containing Tween-80 and EDTA to obtain homogeneous IFN alpha 2b.

The total protein content was determined according to the Bradford's method. Determination of specific protein, IEN alpha 2b was carried out by gel densitometry using Strategene Eagle Eye Video documentation system. Purity of IFN alpha 2b protein was determined by RP-HPLC using YMC protein RP column.

If desired, the highly purified human interferon alpha 2b may be further used for making crystals, to prepare depot formulations [Reichert P., et al., U.S. Pat. No. 5,741,485 (1998);]. Until date, two forms of crystalline human interferon alpha 2 have been reported [Nagabhushan T. L. and Trotta P. P., Ullmaun's Encyclopedia of Industrial Chemistry, A14, VCH, Weinheim, Federal Republic of Germany 372-374 (1989); Miller D. L., et al., Science. 215, 689-690 (1982); Weissmann C., Interferon, Ion Gresser, ed., Academic Press, New York, 101-134 (1981); Nagabhusban T. L., et al., Interferon: Research Clinical Application and Regulatory Consideration, Zoon, et al., eds., Elsevier, NY, 79-88 (1982).]. These publications describe methods for crystallizing interferon alpha-2 from polyethylene glycol at low temperature or from a phosphate buffer solution by adjusting the pH or temperature.

The purified protein of the present invention has been characterized for its physicochemical, immunological and biological characteristics as follows:

Molecular Weight and Purity Determination by SDS-PAGE:

The said protein has been resolved on SDS-PAGE and stained with coomassie blue as described by Oakley et al [150]. The results are summarized in FIG. 7 demonstrating its purity and molecular weight to be ˜19.268 kDa.

Isoelectro Focussing of IFN Alpha 2b Protein (FIG. 9):

The pI of IFN alpha 2b protein of the present invention was determined by IEF (Oakley B. R., et al., Anal. Biochem., 105: 361 (1980)]) and was found to be ˜5.3 which is the expected pI range of human IFN alpha 2b protein (Ref. Methods in Enzymology Vol. 119; 1986 “Interferon standards and general abbreviations.” S. Pestaka.).

LCMS of IFN Alpha 2b Protein (FIG. 10):

To determine the Mol. Wt. of the IFN alpha 2b protein of the present invention, LCMS analysis was carried out & the Mol. Wt. was found to be 19.268 KD, whereas the molecular weight of IFN 2b European reference protein is 19.267 kDa. (Gressen I ed., “Interferons, 1979” Academic Press, New York;).

Utility of the Protein

Type I interferons exhibit potent antiviral properties. Type I interferons also exhibit potent anticellular proliferation activity and immunomodulatory activity. IFN alpha have shown to inhibit various types of cellular proliferation. IFN.alpha.'s are especially useful against hematologic malignancies such as hairy-cell leukemia (Quesada, et al., 1984). Further, these proteins have also shown activity against multiple myeloma, chronic lymphocytic leukemia, low-grade lymphoma, Kaposi's sarcoma, chronic myelogenous leukemia, renal-cell carcinoma, urinary bladder tumors and ovarian cancers (Bonnem, et al., 1984; Oldham, 1985). The role of interferons and interferon receptors in the pathogenesis of certain autoimmune and inflammatory diseases has also been investigated (Benoit, et al., 1993). IFN.alphas are also useful against various types of viral infections (Finter, et al., 1991). Alpha interferons have shown activity against human papillomavirus infection, Hepatitis B, and Hepatitis C infections (Finter, et al., 1991; Kashima, et al., 1988; Dusheiko, et al., 1986; Davis, et al., 1989).

It has also been reported that interferons may be used to treat autoimmune, inflammatory, proliferative and hyperproliferative diseases, as well as cutaneous manifestations of immunologically mediated diseases. In particular, methods of the present invention are advantageous for treating conditions relating to immune system hypersensitivity [Johnson H. M., et al., U.S. Pat. No. 6,204,022 (2001).].

Autoimmune diseases particularly amenable for treatment using the methods of the present invention include multiple sclerosis, type I (insulin dependent) diabetes mellitus, lupus erythematosus, amyotrophic lateral sclerosis, Crohn's disease, rheumatoid arthritis, stomatitis, asthma, uveitis, allergies and psoriasis [Johnson H. M., et al., U.S. Pat. No. 6,204,022 (2001).].

Interferon alpha 2b of the present invention can be used, either singularly or in combination with other therapies as is known in the art for the treatment of any of the above mentioned therapeutic conditions.

Pharmaceutical Compositions

IFN alpha 2b of the present invention can be formulated according to known methods for preparing pharmaceutically useful compositions. Formulations comprising interferons have been previously described (for example, Martin, 1976). In general the compositions of the present invention will be formulated such that an effective amount of the interferon is administered.

The compositions used in these therapies may also be in a variety of forms. These include, for example, solid, semi-solid, and liquid dosage forms, such as tablets, pills, powders, liquid solutions or suspensions, liposomes, suppositories, injectable, and infusible solutions. The preferred form depends on the intended mode of administration and therapeutic application. The compositions also preferably include conventional pharmaceutically acceptable carriers and adjuvants, which are known to those skilled in the art. Preferably, the compositions of the invention are in the form of a unit dose and will usually be administered to the patient one or more times a day.

The IFN.alpha 2b of the present invention may be administered to a patient in any pharmaceutically acceptable dosage form, including but not limited to oral intake, inhalation, intranasal spray, intraperitoneal, intravenous, intramuscular, intralesional, or subcutaneous injection.

Interferon alpha protein solution formulations as mentioned in U.S. Pat. No. 5,766,582 (Schering Plough) describes a process for making stable aqueous solution, formulations containing Alfa-type interferon for e.g. IFN alpha 2a and IFN alpha 2b, a phosphate buffer between pH range 6.6 to 7, Tween-80 as a stabilizer, EDTA as chelating agent, NaCl as a tonicity agent and m-cresol as an antimicrobial agent which maintain high chemical, physical and biological stability of the IFN alpha for an extended storage period of at least 24 months. The same is included in this invention by way of reference.

U.S. Pat. No. 4,496,537 (Schering Corp.) describes a process for formulation of IFN alpha in lyophilized form wherein phosphate buffer of pH 6.8 to 7.0 containing 2.0 mg % glycine and 0.1 gm % HSA is used to lyophilize IFN alpha at concentration of 7.5×10¹⁰ I.U. per litre. Protein was found to be stable retaining biological activity for longer time. This is also incorporated by way of reference in the present invention.

EP 0809996 A2 (Hoffman-La-Roche) describes a process for physiologically active PEG-IFN alpha conjugates and the process for Pegylation of IFN alpha protein which are also included as reference.

The present invention, thus generally described, will be understood more readily by reference to the following examples, which are provided by way of illustration and are not intended to be limiting of the present invention.

### EXAMPLES

In the following examples, Pichia pastoris strain KM 71 was used as host strain, for transformation with the vector. The transformed Pichia host of present invention contained an expression vector prepared from pPICZalpha A (Invitrogen Corporation) which will be referred to as ZBT-alpha A hereinafter. This vector was ligated with modified human interferon alpha 2b gene after double restriction digestion with XhoI+NotI or EcoRI+NotI. The so obtained modified IFN alpha 2b clone was used further for cloning of mature human IFN alpha 2b gene using Xho I+Xba I or Xho I+Not I restriction sites. The fermentation process involved multiple approaches to obtain high-density cell culture described in this document. The IFN alpha 2b protein secreted is isolated and purified using simple techniques as described elsewhere in the document.

### Example 1

### Isolation of Modified Human IFN Alpha 2b Gene

Synthesis of Interferon specific RNA was stimulated in human leukocytes based on the strategies outlined herein and by the procedures described in literature [Cantell K., et al, Methods in Enzymol., Academic Press, N.Y., 78:29-38, (1981); Khavkin T., et al, J. Leukocyte Biology, Annual Meeting Abstracts, Suppl. 3, Abstr. 137:36 (1992); Wheelock E. F., J. Bact. 92, 1415-1421, (1966);]. mRNA was isolated and purified by using oligo dT columns according to known methods [Hiscott I., et al, Nucl. Acids. Res., 12, 3727-3746 (1984)]. The purified mRNA was used to prepare first strand of DNA by RT-PCR technique. The reaction mixture contained 500 nanograms mRNA, 50 units RNAse inhibitor, 20 units AMV reverse transcriptase, dNTP mix and oligo dT 17 in a 20 μl reaction volume and was incubated at 42° C. for 60 min. In next step double stranded DNA was prepared using novel primers which are as described in Table 2, preferably with seq. ID 4+5. or 6+0.7 The reaction mixture consists of cDNA synthesized above, along with 150 nM of each primer (SEQ ID 4+5 or 6+7), 150 μM of dNTP mixture and 1.5 mM MgCl₂ in PCR reaction buffer and PCR amplification was carried as usual. The product obtained was resolved on 1% agarose gel containing ethidium bromide in 1×TAE buffer at 50 V for 2 hrs.

TABLE 2
Primers for PCR Amplification:

| SEQ. | | | Length |
|---|---|---|---|
| ID NO | Direction | Primer | (bases) |
| 4 | Forward | 5′-ATGGCCTTGACCTTTGCTTTACT-3′ | 29 |
| 5 | Reverse | 5′-TCATTCCTTACTTCTTAAACTTTCTTGCA-3′ | 29 |
| 6 | Forward | 5′GAAGCGGAGGCTGAATTCTGTGATCTGCCTCAA-3′ | 33 |
| 7 | Reverse | 5′TCATTCCTTACTTCATAAACTTTCTTGCAAG-3′ | 31 |
| 8 | Forward | 5′ATCTCGAGAAAAGAGAAGCGGAGGCTGAATTCTGTGATCTGCCT-3′ | 44 |
| 9 | Reverse | 5′AAGCGGCCGCTCATTCCTTACTTCTTAAACTTTCT-3′ | 35 |
| 10 | Forward | 5′-GGGAATTCTGTGATCTGCCTCAAA-3′ | 24 |
| 11 | Reverse | 5′-TTGCGGCCGCTCATTCCTTACTT-3′ | 23 |
| 12 | Forward | 5′ ATC TCG AGA AAA GAT GTG ATC TGC CTC AA 3′ | 29 |
| 13 | Reverse | 5′ TAT TCT AGA TCA TTC CTT ACT TCT TAA 3′ | 27 |
| 14 | Reverse | 5′ AAG CGG CCG CTC ATT CCT TAC TTC TTA A 3′ | 28 |

The sequence of forward and reverse primers is given in 5′ to 3′ direction.

### Example 2

### Cloning of Modified IFN Alpha 2b Gene in E. coli JM109:

The M13mp18 plasmid found in E. coli was isolated from 2.0 ml of overnight cultures grown at 37° C. using known method [Westermeier, R. Electrophoresis in Practice, 2^{nd} Ed., VCR, Winheim, Germany (1997).]. The plasmid DNA was resolved on 1.5% agarose gel and quantified. The size of the inserts was determined by digestion with restriction endonucleases Hinc II and later was verified for purity and quantified. The dephosphorylated linearized M13 mp18 plasmid was ligated with the cDNA obtained in Example 1. The ligation reaction contained the above two in 1:3 ratio and 3 units of T4 DNA ligase, 1× ligation buffer and 1 mM riboATP in 20 μl of reaction mixture. This ligated construct was transformed in E. coli JM 109 competent cells by CaCl₂ method [Ref. Methods in Enzymology Vol. 119; 1986 “Interferon standards and general abbreviations.” S. Pestaka.]. The transformants were grown on Luria agar containing X-gal and IPTG, from which 20 white recombinant plaques were isolated and named as GAS 8W1 through GAS 08W20,GAS being the code given for modified IFN alfa 2b gene of the present invention.

The RF DNA was isolated from recombinant E. coli (GAS 08W2) and 2 to 5 μg of RF DNA was subjected to double restriction digestion. The reaction mixture had 2 units each of EcoR1 and Hind III restriction enzymes, 1× universal buffer in 50 μl solution and was incubated at 37° C. for 4 to 5 hrs. The product was resolved on 1% agarose gel having 0.5 μg/ml of ethidium bromide. The size of the released fragment was confirmed as ˜580 bp when compared with standard molecular weight markers. This fragment henceforth was called GAS 2b gene, was purified by using QIA quick gel extraction kit (Qiagen) as per manufacturer's instructions.

The purified fragment was sequenced using Sanger's dideoxy chain termination method with the fluorescent dye chemistry. DNA sequence is as shown in FIG. 1. (SEQ ID Nos. 1).

### Example 3

### Cloning of Modified IFN Alpha 2b Gene in E. coli TOP 10F′

The expression vector pPICZ alpha A, was obtained from Invitrogen Corporation (here after called ZBT alpha A) and propagated in E. coli TOP 10F′. The plasmid DNA was isolated by alkali lysis method [Westermeier, R. Electrophoresis in Practice, 2d Ed., VCH, Winheim, Germany (1997)]. The modified GAS 2b gene was amplified using primers, having seq. ID Nos. 8 & 9 and 10 & 11 as forward & reverse primers, which are described in Table 2. The GAS 2b gene from GAS 08W2 DNA was reamplified. The reaction mixture contained 100 nM of template GAS 08W2 DNA, 1×PCR buffer, 1.5 mM MgCl₂, 150 μM dNTP mixture, 150 nM of each primer and 2 units of Taq polymerase (MBI Fermentas) according to previously described procedure. An aliquot of amplified DNA was resolved on 1.5% agarose gel containing 0.5 μg/ml of ethidium bromide along with the standard 1 kb ladder marker (MBI Fermentas). Two to five μg of PCR product from each reaction were restriction digested in reaction containing 2-5 units each of Xho I+Not I; EcoRI+Not I; enzymes respectively, in 1× universal buffer in a 50 μl solution. The sample was resolved on agarose gel having 0.5 μg/ml of ethidium bromide. The size of the released fragment was confirmed as ˜516 bp when compared to standard molecular weight markers in the adjacent lane.

Similarly, the ZBTalpha A vector was restriction digested (in separate reactions) using Xho I+Not I; EcoRI+Not I, 1× universal buffer in 50 μl solution.

The purified cDNA was ligated to ZBTalpha A vector DNA. This ligated DNA was then used to transform E. Coli TOP 10F′ by electroporation method. The competent E coli cells were mixed with 100 ng of purified ligated DNA in chilled electroporation cuvette and cells were transformed using Electroporetor 1000 (Strategene) [Gressen I ed., “Interferons, 1979” Academic Press, New York;]. The transformed cells were plated on a low salt Luria agar containing 25 μg/ml each of zeocin and tetracycline, plates were incubated at 37° C. overnight. The transformants, (hereinafter called E coli ZBT-IFMB 1, 2, 3 - - - ) were verified for the presence of the modified GAS 2b gene as described before. The orientation of the GAS 2b gene was confirmed by using the combination of vector specific and gene specific primers. The positive clones or the recombinant clones having the modified IFN alpha 2b gene were called E coli ZBT-IFMBI, 2, 3. and E coli ZBT-IFMB3 clone was sequenced using the dideoxy termination method. The sequence data was of modified interferon alpha 2b gene {FIG. 1—SEQ ID No. 1}.

### Example 4

### Cloning of IFN Alpha 2b Gene in E coli TOP 10 F′

The modified IPN alpha 2b clone ZBT-IF MB3 of EXAMPLE 3 was further used for cloning of human interferon alpha 2b of this invention. Plasmid DNA was isolated from the clone using Wizard Plus SV Miniprep DNA purification system (Promega). This DNA was then restriction digested using Xho I+Not I or EcoRI+Not I enzymes, 1× universal buffer in 20 μl solution to release the cloned modified IFN alpha 2b fragment. This fragment was purified from the gel using QIAquick Gel Extraction Kit (QIAGEN) and used as a template for PCR amplification of human IFN alpha 2b of this invention with Primers having SEQ ID 12 & 13 or SEQ ID 12 & 14 as forward and reverse primers.

The reaction mixture contained the above DNA as template, 1×PCR buffer, 1.5 mM MgCl₂, 150 μM dNTP mixture, 150 nM of each primer and 3 units of Taq polymerase (MBI Fermentas) according to previously described procedure. An aliquot of amplified DNA was resolved on 1.0% agarose gel containing 0.5 μg/ml of ethidium bromide along with the standard 1 kb ladder marker (MBI Fermentas).

Two to five μg of PCR product from the reaction was restriction digested in reaction containing 2-5 units each of Xho I+Xba I or Xho I+Not I enzymes in 1× universal buffer in a 20 μl solution. The sample was resolved on agarose gel having 0.5 μg/ml of ethidium bromide. The size of the amplified and digested product was confirmed as ˜498 bp when compared to standard molecular weight markers in the adjacent lane. (FIG. 3)

Similarly, ZBTalpha A vector was restriction digested (in separate reactions) using Xho I+Xba I or Xho I+Not I enzymes, 1× universal buffer in 20 μl solution. (FIG. 3).

The purified and digested DNA was ligated to ZBTalpha A vector DNA. This ligated DNA was then used to transform E. coli TOP 10F′ by electroporation method. The competent E coli cells were mixed with ˜100 ng of purified ligated DNA in chilled electroporation cuvette and cells were transformed using Electroporetor 1000 (Strategene) [Gressen I ed., “Interferons, 1979” Academic Press, New York;].

The transformed cells were plated on a low salt Luria agar containing 25 μg/ml each of Zeocin and Tetracycline, plates were incubated at 37° C. overnight. The transformants, (hereinafter called E coli ZBT-IF 2.1, 2.2, 2.3 - - - ) were verified for the presence of the IFN alpha 2b gene as described before. (FIGS. 4,5)

The orientation of the IFN alpha 2b gene was confirmed by using the combination of vector specific and gene specific primers. The positive transformant having the right orientation was used for further sub cloning in Pichia pastoris KM71 stain. The positive clone or the recombinant clone having the IFN alpha 2b gene was called E coli ZBT-IF 2.2 and this clone was sequenced using the dideoxy termination method. The sequence data confirmed that the gene is novel, interferon alpha 2b gene{FIG. 2b—SEQ ID No. 3}.

The homology search was carried out with sequences at NCBI GenBank database using “BLAST-N” and it was concluded that the gene of present invention had a nearest match (9%) with published human leukocyte interferon alpha gene sequence. However it did not match 100% with the published gene sequence (FIG. 2a—SEQ ID NO. 2). It had two point mutations, one at 57^{th} nucleotide position and the other at 195^{th} nucleotide position {SEQ ID 3}. Thus the gene of the present invention is a novel human IFN alpha 2b gene.

### Example 5

### Transformation of Yeast Pichia pastoris with Expression Construct

Transformation of yeast Pichia pastoris KM 71 was carried out according to method described in literature using LiCl [Higgins D. R., and Cregg J. M., (eds.), Pichia Protocols, Methods in Molecular Biology, Humana Press, (Totowa, N.J.), 103: 249-261 (1998).].

A single isolated colony of Pichia pastoris KM 71 was grown in 10 ml of YPD media (Table IF). The cells were harvested, washed twice with distilled water and suspended in 10 ml of 100 mM LiCl. Competent cells were recovered by brief centrifugation. Yeast pellet was mixed with 240 μl of 50% PEG, 36 μl of 1 M LiCl, 25 μl of 2 mg/ml SS DNA and 50 μl of the linearized insert (1-10 μg) and construct was introduced into yeast cell by heat shock.

The transformed cells were plated on selective agar plates (YPD agar containing Zeocin antibiotic) and incubated for 2 to 4 days at 30° C. Twenty five transformants/clones were selected and PCR analysis was done using their genomic DNA to determine integration of IFN alpha 2b gene into the Pichia genome (FIG. 6). These twenty five transformants/clones were then screened for the production of the IFN alpha 2b protein at shake flask level. These clones were named as Pichia pastoris ZIP clone 2.2/1-2.2/25

### Example 6

### Development of Inoculum Using BGYP Medium for Fermentation Process

50 ml of BGYP medium (composition Table 1) was taken in 250 ml Erlenmeyer flask and was inoculated with 1 ml inoculum of Pichia pasiors ZIF 2.2/14 (as described in example 5) from thawed glycerol stock (stored at −70° C.). The flask was incubated at 30±0.5° C., on rotary shaker at about 210 rpm, with 1″ displacement for 24-48 hours. 2% v/v of the above inoculum was further diluted in 200 ml BGYP medium into a IL flask, which was incubated for about 48-72 hrs on rotary shaker under the similar conditions. The inoculum prepared was used for fermentation and production of desired protein.

### Example 7

### Development of Inoculum Using BGY Medium for Fermentation Process

In another method, the inoculum was developed using BGY medium. To the medium biotin and histidine stock solutions were added (0.4 mg/L of biotin and 8 mg/L of histidine was present in the medium).

The seed stage I was initiated by inoculating 50 ml BGY medium with 1 ml of thawed glycerol stock of Pichia pastoris ZIF 2.2/14 stored at −70° C., under aseptic condition. The flask was incubated at 30±0.5° C. for about 48 hours on rotary shaker at about 210 rpm, with 1″ displacement. After 24-48 hrs of fermentation, the purity of seed was confirmed by microscopy. Later in seed stage 11,200 ml BGY contained in a 1 L Erlenmeyer flask, was inoculated with 2% v/v concentration of cells from seed stage 1. This seeded flask was incubated on rotary shaker and cultivated under similar conditions. The inoculum prepared was used for fermentation to produce IFN alpha 2b protein.

### Example 8

### Fed-Batch Fermentation with Complex Medium

The fermentation process was carried out under submerged aerobic conditions. The fermenter was equipped with automatic pK, temperature and dissolved oxygen controls. In 20 L fermenter, 8 to 10 L of complex medium was prepared, sterilized and inoculated with the inoculum prepared as described in example 6 or 7. The agitation rate varied between 230 to 450 rpm. The aeration rates varied from about 0.4 to 1.0 volume (at about atmospheric pressure and about 25° C.) per volume of ferment per minute of air supplied. The air supplied was mixed with sufficient oxygen whenever required, in order to maintain dissolved oxygen at about 20 to 60% saturation.

The complex media used in the present fermentation process was prepared as described in Table 1A, after sterilization the media was further supplemented with 2 ml/L each of biotin and histidine stock solutions. The stock biotin and histidine solutions were prepared (Table 1), filter-sterilized and stored at +4° C.

The fermentation was carried out at 30° C. at about atmospheric pressure in fed batch protocol, wherein glycerol was added (10 ml/L of complex media) in rate limiting concentration and was later slowly increased. At the end of about 62 to 64 hours of fermentation process, when carbon source was completely utilized, the cells were separated by centrifugation at 6000-8000 g, and resuspended in 10 litre of BMY medium (described in Table 1B). The cells were transferred back to the fermenter, under aseptic conditions. The cells were induced by methanol (50% aqueous solution), wherein the concentration of methanol was maintained between 0.7 to 2.7% v/v, by measuring the methanol content in the ferment by gas chromatography. Multiple induction were carried out The concentration of expressed r-human IFN alpha 2b protein obtained was ˜200 mg/L as monitored by densitometric analysis of commassie blue stained SDS-PAGE gel.

### Example 9

### Modified Fed Batch Fermentation Process 1

Another fermentation process was developed, similar to the protocol described in Example 8, but involved an additional supply of nitrogen source later in the growth stage. A nitrogen source, was added in concentrated form as BY (10×) medium to the fermentation broth (Table 1). Rest of the procedure/protocol was same as given in the above example 8 and was continued until the protein yield reached at least 200 mg/L. The fermentation process was monitored by carrying densitometric analysis of commassie blue stained SDS-PAGE gel of the fermentation samples.

### Example 10

### Modified Fed Batch Fermentation Process 2

A continuous aerobic fermentation process was carried out in a fermenter as described in Example 8, this time instead of complex medium, a defined soluble salt media (Table 1D) was used. In twenty litre fermenter having 10 litre of defined salt medium having composition described in Table 1D was prepared, and trace metal solution A and B were added to this and pH was adjusted to 3.5. The medium was sterilized in fermenter at 121° C. for 30 min. After sterilization, pH of the medium was adjusted to 5.0 with sterile ammonium hydroxide solution and supplemented with biotin and histidine. The fermentation media was inoculated with 2% v/v inoculum developed as described in Example 7.

The culture was stirred continuously by passing air supplemented with sufficient oxygen to maintain dissolved oxygen level at about 20-60% of saturation. Aqueous ammonium hydroxide was also added at a rate so as to maintain the pH of the fermentation mixture at about 3.5 to 4.5. The fermentation was carried out at 30° C. and at atmospheric pressure.

The pH was maintained in the range of 3.5 to 4.5 initially and increased gradually, from about 3.0-3.5 to 4.5-5.0 at the end of fermentation. Glycerol feed was started initially at 1 ml/L/hr rate and later increased to a maximum of up to 20 ml/L/hr. After 62 to 64 hours (including a starvation period of at least two hours), the cells were induced by methanol.

The concentration of methanol was maintained about 0.7 to 2.7% v/v and induction was continued further till proteins were being actively secreted and the protein yield achieved was 200 mg/L.

The fermentation process was monitored by densitometric analysis of coommassie blue stained SDS-PAGE gel of fermentation samples run along with the known standard. It was observed that with present process of fermentation, apart from desired protein another protein having about 20 kDa molecular weight was also secreted.

### Example 11

### Modified Batch Fermentation Process 3

The fermentation process was initiated in a fed batch mode as described in previous example 10, except modification of glycerol feed rate which varied from 0.3 ml/L/hr to 20 ml/L/hr during initial 78-108 hours of fermentation time. The fermentation was conducted at 30° C. and about atmospheric pressure. The pH, dissolved oxygen, agitation rates were maintained similar to that in example 10.

After above-mentioned period, glycerol feed was stopped, fermentation continued for 1-2 hours so as to allow complete utilization of glycerol. Cells were induced as described in example 10. The secreted IFN alpha 2b protein yield was ˜200 mg/L. The process was monitored by recording the amount of methanol in the medium by G.C. and the protein yield was monitored by densitometric analysis of coomassie blue stained SDS-PAGE gel of fermentation samples run along with the known standard. It was observed that as in Example 10 with present process of fermentation, apart from desired protein another protein having about 20 kDa molecular weight was also secreted.

### Example 12

### Modified Batch Fermentation Process 4

The fermentation process was initiated as described in previous example 11. At the end of growth stage and additional starvation period of 0.5-1 hour, 1 liter of 10×BMY was added to the fermenter.

The induction was initiated by addition of filter sterilized methanol solution (50% v/v aqueous methanol containing 6 ml/L each of trace metal solution PTM A and PTM B) to maintain methanol concentration of about 1.5 to 3.0% v/v. Further addition or subsequent induction was started when methanol concentration dropped to about 0.5 to 0.8%. Throughout the production stage pH of fermentation was maintained at around 6.0±0.1 by addition of ammonia solution through pH controller. Levels of desired protein produced was monitored by densitometric analysis of coommassie blue stained SDS-PAGE gels of fermentation samples run along with known standard. The yield of IFN alpha 2b protein was ˜550-600 mg/L of fermentation broth. It was observed that with present process of fermentation, apart from desired protein another protein having about 20 kDa molecular weight was also secreted.

### Example 13

### Modified Batch Fermentation Process 5

The fermentation process was initiated in a fed batch mode as described in previous example 10, except modification of glycerol feed rate which varied from 0.3 ml/L/hr to 20 ml/L/hr during initial 78-108 hours of fermentation time. The fermentation was conducted at 30° C. and about atmospheric pressure. The pH, dissolved oxygen, agitation rates were maintained similar to that in example 10.

At the end of growth stage, the cells were separated by centrifugation at 6000-8000 g, and resuspended in 10 liter of BMY medium (described in Table 1B). The cells were transferred back to the fermenter, under aseptic conditions. The cells were induced by methanol (50% aqueous sample was taken for SDS-PAGE and RP-HPLC analysis. Single band purity observed on SDS-PAGE (Coomassiae blue stain) analysis and the purity obtained was 97.25% by RP-HPLC.

### Example 15

### Purification of Recombinant IFN Alpha 2b Protein to Homogeneity

Step A: The supernatant containing recombinant IFN alpha 2b protein was separated from the fermented broth by centrifugation at 6000-8000 g for 10 min at 4-8° C. Supernatant was cooled to 8-10° C., and diluted 1:1 volume with Milli Q water. The pH of solution was adjusted to 5.2 and was filtered through 0.45 micron cartridge (Milliguard, Millipore Inc.) at low temperature.

Step B: In the next step, Super flow radial 1500 column (Sepragen Inc.) was packed with weak cation exchanger CM Sepharose FF or SP Sepharose FF (Amersham Biosciences) (Bed volume 1500 ml) and pre-equilibrated with chilled CEX buffer I (50-100 mM Ammonium Acetate, pH=4.5-5.5, preferably 4.8-5.3 adjusted with acetic acid). Reconditioned chilled supernatant obtained in step A, was loaded on the column. After capturing the desired protein on CM Sepharose column, the column was washed with CIEX buffer I followed by desired protein elution with CIEX buffer II (500 mM Ammonium Acetate pH=4.5-5.7, preferably 4.8-5.4 adjusted with acetic acid). All the steps were carried out at low temperature (10 to 12° C.) and a sample was taken for analysis.

Step C: The eluant obtained in step B, which is containing IFN alpha 2b protein, was diluted 10 times with Milli Q water and pH was adjusted to 6.3-7.3. This material was directly loaded on pre-equilibrated anion exchange column (DEAE Sepharose FF or Q Sepharose FF (Amersham Biosciences)). After capturing the desired protein onto DEAE Sepharose column, the column washed with AIEX buffer I (50-100 nM Ammonium Acetate, pH=6.3-7.3, preferably 6.5-7.0 adjusted with acetic acid/liquor ammonia) followed by elution of the desired protein using AEEX buffer II (150-250 mM Ammonium Acetate, pH=5.3-6.8, preferably 5.5-6.5 adjusted with acetic acid). All the steps were carried out at low temperature (10 to 12° C.) and a sample was taken for analysis.

Step D: To the eluant obtained in step C, which contains IFN alpha 2b protein, was added ammonium sulfate to achieve a final concentration of 0.75 M-1.5 M, preferably 0.75 M to 1.2 M of ammonium sulfate and pH was adjusted to 6.3-6.8, more preferably 6.5-6.8. The solution containing IFN alpha 2b protein was filtered through 0.45 micron disc filter. The filtrate was directly loaded on pre-equilibrated HIC column (XK 50/200 mm) (Butyl Sepharose FF or Butyl Toyo (Amersham Biosciences/Toshohaas)). After capturing the desired protein onto the HIC column, the column was washed with HIC buffer 1 (10-20 mM Sodium phosphate containing Ammonium sulphate at concentration of 0.75-1.2 M, pH=6.3-6.8, preferably 6.5-6.8 adjusted with phosphoric acid/NaOH) followed by elution of the desired protein using EHC buffer II (10-20 mM Sodium phosphate, pH=6.5-6.8 adjusted with phosphoric acid/NaOH). All the steps were carried out at low temperature (10 to 12° C.) and a sample was taken for analysis.

Step E: The eluant from step D containing IFN alpha 2b, was concentrated 20 times by ultrafiltration using Amicon Stirred Cell (400 ml capacity) with YM 10 membrane (10,000 MWCO). The final concentration of IFN alpha 2b protein in retentate was ˜15-20 mg/ml.

Step F: The gel filtration column was packed with Sephacryl HR 200/HR 100/Sephadex G 75/Sephadex G 25 cores (Pharmacia), equilibrated with gel filtration buffer (10 mM Ammonium acetate, containing 150 mM NaCl, 0.2% w/v EDTA, 0.2% v/v Tween −80, and pH was adjusted to 5.0-5.5 with acetic acid). The concentrated IFN alpha 2b protein obtained in step E was loaded on this column. An isocratic elution was carried out using gel filtration buffer. The peak fraction containing pure homogenous IFN alpha 2b protein was collected and a sample was taken for SDS-PAGE analysis. Single band purity observed on SDS-PAGE (Coomassiae blue stain) analysis.

### Example 16

### Purification of Recombinant IFN Alpha 2b Protein to Homogeneity

Step A: The supernatant containing recombinant IFN alpha 2b protein was separated from the fermented broth by centrifugation at 6000-8000 g for 10 min at 4-8° C. Supernatant was cooled to 8-10° C., and diluted 1:1 volume with Milli Q water. The pH of solution was adjusted to 5.2 and was filtered through 0.45 micron cartridge (Milliguard, Millipore Inc.) at low temperature.

Step B: In the next step, Super flow radial 1500 column (Sepragen Inc.) was packed with weak cation exchanger CM Sepharose FF or SP Sepharose FF (Bed volume 1500 ml) and pre-equilibrated with chilled CIEX buffer 1 (50 mM Ammonium Acetate+2.5 mMol. EDTA, pH 4.5-5.3 adjusted with acetic acid). Reconditioned chilled supernatant obtained in step A, was loaded on the column. After capturing the desired protein on CM Sepharose column, the column was washed with CIEX buffer I followed by desired protein elution with CIEX buffer II (500 mM Ammonium Acetate+2.5 mMol. EDTA, pH=4.3-5.5 adjusted with acetic acid). All the steps were carried out at low temperature (10 to 12° C.) and a sample was taken for analysis.

Step C: The eluant obtained in step B, which is containing IFN alpha 2b protein, was diluted 10 times with Milli Q water and pH was adjusted to 6.80. This material was directly loaded on pre-equilibrated on anion exchange column (DEAE Sepharose FF). After capturing the desired protein onto DEAE Sepharose column, the column washed with AIEX buffer I (50 mM Ammonium Acetate+2.5 mMol. EDTA, pH=6.0-7.3 adjusted with acetic acid) followed by elution of the desired protein using AIEX buffer II (150 mM Ammonium Acetate+2.5 mMol. EDTA, pH=5.3-5.8 adjusted with acetic acid). All the steps were carried out at low temperature (10 to 12° C.) and a sample was taken for analysis.

Step D: The eluant from step C containing IFN alpha 2b, was concentrated 20 times by ultrafiltration using Amicon Stirred Cell (400 ml capacity) with YM 10 membrane (10,000 MWCO). The final concentration of IFN alpha 2b protein in retentate was ˜15-20 mg/ml.

Step E: The Versaflo Axial 9/60 Cm column (Sepragen Inc.) was packed with Sephacryl HR 200 (Pharmacia), bed volume 3.7 liter and equilibrated with gel filtration buffer (10 mM Ammonium acetate, containing 150 mM NaCl, 0.2% w/v EDTA, 0.2% v/v Tween −80, and pH was adjusted to 5.5 with acetic acid). About 45 ml of concentrated IFN alpha 2b protein obtained in step D was loaded on this column. An isocratic elution was carried out using gel filtration buffer. The peak fraction containing pure homogenous IFN alpha 2b protein was collected and a sample was taken for SDS-PAGE analysis. Single band purity was observed on SDS-PAGE (Coomassiae blue stain) analysis.

### Example 17

### Purification of Recombinant IFN Alpha 2b protein to Homogeneity

Step A: The supernatant containing recombinant IFN alpha 2b protein was separated from the fermented broth by centrifugation at 6000-8000 g for 10 min at 4-8° C. Supernatant was cooled to 8-10° C., and diluted 1:1 volume with Milli Q water. The pH: of solution was adjusted to 5.2 and was filtered through 0.45 micron cartridge (Milliguard, Millipore Inc.) at low temperature.

Step B: In the next step, Super flow radial 1500 column (Sepragen Inc.) was packed with weak cation exchanger CM Sepharose FF or SP Sepharose PF (Amersham Biosciences) (Bed volume 1500 ml) and pre-equilibrated with chilled CIEX buffer I (50-100 mM Ammonium Acetate+2.5 mMol. EDTA, pH=4.5-5.5, preferably 4.8-5.3 adjusted with acetic acid). Reconditioned chilled supernatant obtained in step A, was loaded on the column. After capturing the desired protein on CM Sepharose column, the column was washed with CIEX buffer I followed by desired protein elution with CIEX buffer II (150 mM Ammonium Acetate+2.5 mMol. EDTA, pH=4.5-5.7, preferably 4.8-5.4 adjusted with acetic acid). All the steps were carried out at low temperature (10 to 12° C.) and a sample was taken for analysis.

Step C: The eluant obtained in step B, which is containing IFN alpha 2b protein, was diluted 10 times with Milli Q water and pH was adjusted to 6.3-7.3. This material was directly loaded on pre-equilibrated on anion exchange column (DEAE Sepharose FF or Q Sepharose FF (Amersham Biosciences)). After capturing the desired protein onto DEAE Sepharose column, the column washed with AIE-X buffer I (50-100 mM Ammonium Acetate+2.5 mMol. EDTA, pH=6.3-7.3, preferably 6.5-7.0 adjusted with acetic acid/liquor ammonia) followed by elution of the desired protein using AIEX buffer II (150-250 mM Ammonium Acetate+2.5 mMol. EDTA, pH=5.3-6.8, preferably 5.5-6.5 adjusted with acetic acid). All the steps were carried out at low temperature (10 to 12° C.) and a sample was taken for analysis.

Step D: To the eluant obtained in step C, which contains IFN alpha 2b protein, was added ammonium sulfate to achieve a final concentration of 0.75 M-1.5 M, preferably 0.75 M to 1.2 M of ammonium sulfate and pH was adjusted to 6.3-6.8, more preferably 6.5-6.8. The solution containing IFN alpha 2b protein was filtered through 0.45 micron disc filter. The filtrate was directly loaded on pre-equilibrated HIC column (XK 50/200 mm) (Butyl Sepharose FF or Butyl Toyo (Amersham Biosciences/Toshohaas)). After capturing the desired protein onto the HIC column, the column was washed with HIC buffer I (10-20 mM Sodium phosphate containing Ammonium sulphate at concentration of 0.75-1.2 M, pH=6.3-6.8, preferably 6.5-6.8 adjusted with phosphoric acid/NaOH) followed by elution of the desired protein using HIC buffer II (10-20 mM Sodium phosphate, pH=6.5-6.8 adjusted with phosphoric acid/NaOH). All the steps were carried out at low temperature (10 to 12° C.) and a sample was taken for analysis.

Step E: The eluant from step D containing IFN alpha 2b, was concentrated 20 times by ultrafiltration using Amicon Stirred Cell (400 ml capacity) with YM 10 membrane (10,000 MWCO). The final concentration of IFN alpha 2b protein in retentate was ˜15-20 mg/ml.

Step F: The gel filtration column was packed with Sephacryl HR 200/HR 100/Sephadex G 75/Sephadex G 25 cores (Pharmacia), equilibrated with gel filtration buffer (10 mM Ammonium acetate, containing 150 mM NaCl, 0.2% w/v EDTA, 0.2% v/v Tween −80, and pH was adjusted to 5.0-5.5 with acetic acid). The concentrated IFN alpha 2b protein obtained in step E was loaded on this column. An isocratic elution was carried out using gel filtration buffer. The peak fraction containing pure homogenous IFN alpha 2b protein was collected and a sample was taken for SDS-PAGE analysis. Single band purity was observed on SDS-PAGE (Coomassiae blue stain) analysis.

1. A process for preparation and purification of recombinant human interferon (hu-IFN) alpha 2b which comprises:
I. cultivating recombinant Pichia pastoris containing a hu-IFN alpha 2b gene,
II. culturing said recombinant Pichia pastoris in complex/defined salt culture medium to produce hu-IFN alpha 2b protein, and
III. purifying recombinant hu-IFN alpha 2b protein from said culture medium.

2. A process as claimed in claim 1 wherein said human IFN alpha 2b gene comprises (SEQ ID NO:3).

3. A process as claimed in claim 1 wherein said recombinant Pichia pastoris containing a hu-IFN alpha 2b gene is cultivated by first isolating and purifying mRNA from human leucocytes, preparing a first strand of DNA from said purified mRNA to obtain said modified hu-IFN alpha 2b gene, amplifying said gene and cloning said amplified modified hu-IFN alpha 2b gene into an expression vector, amplifying and isolating said hu-IFN alpha 2b gene from said modified interferon alpha 2b cloned cloning said hu-IFN alpha 2b gene into an expression vector and transforming said expression vector into said Pichia pastoris.

4. A process as claimed in claim 1 wherein said cloning is carried out by RT-PCR methods employing primer pairs having the sequence selected from the group consisting of SEQ ID NOS: 4 & 5; 6 & 7; 8 & 9; 10 & 11; 12 & 13 and 12 & 14.

5. A process as claimed in claim 3 wherein said Pichia pastoris is selected from Pichia pastoris KM 71, Picha pastoris KM 71H. Pichia pastoris GS115. Pichia pastoris X33 preferably Pichia pastoris KM71.

6. A process as claimed in claim 3 wherein said vector is pPICZαA.

7. A process as claimed in claim 6 wherein said hu-IFN alpha 2b gene is cloned in pPICZαA vector down stream to AOX promoter and alpha mat signal sequence.

8. A process as claimed in claim 3 wherein a desired construct containing a hu-IFN alpha 2b gene (expression cassette) is integrated at the AOX region of Pichia pastroris.

9. A process as claimed in claim 8 wherein said expression cassette is integrated at the 5′ AOX region of Pichia pastoris selected from Pichia pastoris KM 71, Picha pastoris KM 71H, Pichia pastoris GS115, Pichia pastoris X33 preferably Pichia pastoris KM71.

10. A process as claimed in claim 9 wherein said Pichia pastoris has His auxotrophic phenotype.

11. A process as claimed in claim 1 wherein said culture medium is selected from complex media like BGY, BY, BMY, BGYP, YPD, defined salt medium preferably defined salt medium and BMY.

12. A process as claimed in claim 11 wherein said culture medium comprises one or more nitrogen sources selected from the group consisting of ammonium salts, nitrates, corn steep liquor, peptone, casein, meat extracts, bean-cakes, potato extracts, protein hydrolysates, yeast extract, urea and ammonium hydroxide.

13. A process as claimed in claim 1 wherein said culture medium comprises a carbon source such as glycerol, glucose, fructose, methanol and the like, preferably glycerol.

14. A process as claimed in claim 1 wherein the biomass build up is in a range of 35 to 100 g/L, preferably 35-50 g/L for complex medium media and 50 to 80 g/L, preferably 50-60 g/L for defined salt medium based on dry cell weight.

15. A process as claimed in claim 14 wherein said culture medium has:
(a) pH in the range of 3.0 to 6.0, preferably 6.0 to 6.5 for complex medium and 3.5 to 4.5 for defined salt medium preferably 5.8 to 6.2,
(b) temperature in the range of 25 to 35° C. preferably 28 to 32° C., and
(c) dissolved oxygen: 20-80% of saturation, preferably 40-50% of saturation and said culturing is carried out for a duration of 48 to 110 hours, preferably 48 to 72 hours for complex medium and 90-110 hours for defined salt medium.

16. A process as claimed in claim 11 wherein the expression of recombinant IFN alpha 2b protein is induced after reaching appropriate biomass buildup using suitable alcohol such as methanol, ethanol and the like preferably methanol at concentration of 0.1 to 3.0% v/v, preferably 1-1.5% v/v.

17. A process as claimed in claim 16, wherein the expression of full length recombinant IFN alpha 2b protein is regulated by addition of nitrogen source selected from yeast nitrogen base, yeast nitrogen base without amino acid, yeast hydrolysate, yeast extract, peptone, casamino acid, meat extract, beef extract and like, preferably yeast extract and peptone along with or without propylene glycol.

18. A process as claimed in claim 1 wherein said recombinant hu-IFN alpha 2b protein is purified to homogeneity by
(a) separating the cells from the cell culture to obtain the supernatant which contains recombinant hu-IFN alpha 2b protein,
(b) subjecting said supernatant to cation exchange chromatography by
(i) binding said recombinant hu-IFN alpha 2b protein on a column packed with CM SEPHAROSE FF, SP SEPHAROSE FF or SEPRAPREP S,
(ii) washing said column with a buffer selected from citrate, phosphate, acetate buffer or CIEXI buffer, at a pH 5.0-5.5 to remove unwanted proteins, and
(iii) eluting said recombinant hu-IFN alpha 2b protein with CIEXII buffer with pH 4.8-5.4;
(c) subjecting the eluent obtained in step (b)(iii) to anion exchange chromatography followed by elution with AIEX II buffer;
(d) subjecting the eluent from step (c) to ultrafiltration with membrane of pore size 10,000 Dalton molecular cut off to obtain a concentrated retentate containing recombinant hu-IFN alpha 2b protein;
(e) subjecting said concentrated retentate to gel filtration chromatography using ammonium acetate buffer containing Tween-80 and EDTA, pH 5.2-5.5, to obtain homogenous species of recombinant hu-IFN alpha 2b protein; and
(f) purifying said recombinant hu-IFN alpha 2b protein obtained in step (e) by repeating steps (a) to (e) in any sequence or order.

19. A pharmaceutical composition comprising purified interferon alpha 2b prepared and purified according to claim 1, and a pharmaceutically acceptable carrier either in liquid form or in lyophilized form.

20. A pharmaceutically composition as claimed in claim 19 wherein said pharmaceutically acceptable carrier comprises phosphate buffer, glycine, HSA, PEG, ammonium acetate, NaCl, Tween-80, EDTA, Benzyl alcohol and the like in any combination and with desired concentration/amount.

21. A method of treatment using purified interferon alpha 2b prepared and purified according to claim 1 for treatment of viral diseases like chronic active Hepatitis B, Chronic active Hepatitis non A-non B, Chronic active Hepatitis delta, Chronic active Hepatitis C; cancer diseases like Chronic myelogenous leukemia, Non-Hodgkin's lymphoma, AIDS related Kaposi's Sarcoma, Renal cell carcinoma, Malignant melanoma, Hairy cell leukemia, Bladder carcinoma, Superficial and noduloulcerative basal cell carcinoma, Condylomata acuminata, Laryngeal papillomatosis, and like.
